(19) United States
(12) Patent Application Publication
(10) Pub. No.: US 2021/0340163 A1
(43) Pub. Date: Nov. 4, 2021

(54) BORONDIFLUORIDE COMPLEXES OF CURCOMINOID COMPOUNDS, METHOD OF PREPARATION AND USES THEREOF

(71) Applicants: UNIVERSITE D'AIX MARSEILLE, Marseille (FR); CENTRE NATIONAL DE LA RECHERCHE SCIENTIFIQUE, Paris (FR)

(72) Inventors: Elena ZABOROVA, Marseille (FR); Frédéric FAGES, Sanary Sur Mer (FR); Anthony D'ALEO, Marseille (FR)

(73) Assignees: Universite d'Aix Marseille, Marseille (FR); Centre National de la Recherche Scientifique, Paris (FR)

(21) Appl. No.: 15/777,365

(22) PCT Filed: Nov. 8, 2016
(86) PCT No.: PCT/EP2016/077013

§ 371 Date: May 18, 2018

(30) Foreign Application Priority Data
Nov. 24, 2015 (EP) 15306858.0

(51) Int. Cl. C07F 5/02

(52) U.S. Cl. CPC C07F 5/022

(57) ABSTRACT

The present invention relates to new borondifluoride complexes of curcuminoid compounds with an enhanced fluorescence quantum yield and emission, and their uses as fluorophore in various fields such as bioimaging, therapeutics, theranostics, display and telecommunication technologies, photovoltaics. The preparation said compounds is also described.

The present invention relates to new borondifluoride complexes of curcuminoid compounds with an enhanced fluorescence quantum yield and emission, and their uses as fluorophore in various fields such as bioimaging, therapeutics, theranostics, display and telecommunication technologies, photovoltaics. The preparation said compounds is also described.

Compounds displaying high molar absorption coefficients, large two-photon absorption cross sections, high luminescence quantum yields in solution and in the solid state, large Stokes shifts, high thermal and photochemical stability represent attractive candidates as fluorescent reporters. Especially, dyes that emit in the near infrared (NIR, wavelengths longer than 700 nm according to The International Commission on Illumination) using two-photon excitation in the NIR region are of great interest for use in cell imaging because both excitation and detection operate in the biological transparency window.

Among the many classes of organic dyes, boron complexes, such as borondipyrromethene (BODIPY) compounds, are particularly attractive. In addition of the interesting optical properties in solution, many borondifluoride complexes (other than BODIPY) have also been shown to yield rather efficient photoluminescent behavior in the solid state. In particular, compounds deriving from acetylacetonate ligand have shown interesting properties such as high two-photon absorption cross sections, mechano-fluorochromic behaviors and efficient NIR emissions that led to their use for cells imaging or as sensors of volatile acid/base, fluorescent reporters for amyloid, optical sensors for anaerobic environment and electron donors in solar cells.

Recently, curcuminoid structures containing the borondifluoride unit have been shown as efficient emitters that fulfill many of the previous requirements. However, this work also led to the conclusion that solid-state fluorescence of BF₂ complexes of curcuminoids arose from highly stacked chromophores. Such interactions could explain the emission occurring in the NIR but inherently induce efficient face-to-face quenching, which limit the fluorescence quantum yield (Φ_{f}) to ca. 5% (A. D'Aléo, D. Gachet, V. Heresanu, M. Giorgi and F. Fages, Chem. Eur. J, 2012, 18, 12764-12772; G. Bai, C. Yu, C. Cheng, E. Hao, Y. Wei, X. Mu and L. Jiao, Org. Biomol. Chem., 2014, 12, 1618-1626).

There exists a real need for new compounds with improved absorption ability and fluorescence quantum yield in solution and in the solid state.

It is an aim of the present invention to specifically meet these needs by providing a compound of general formula (I):

Q²-Q^{L}Q³ (I)

wherein

Q¹ represented by formula (II)

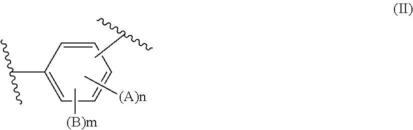

wherein
   Q¹ is substituted with Q² and Q³,
   A is a C₁-C₁₂ alkoxy group,
   B is a difluoroboron beta-diketone of formula (III)

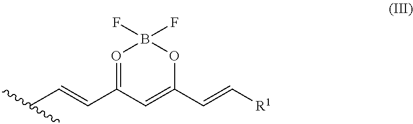

n is 0, 1, 2, 3 or 4,
m is 0 or 1; or

Q¹ is represented by formula (IV)

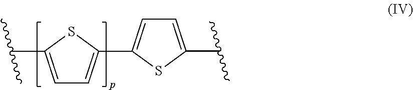

wherein
   Q¹ is substituted with Q² and Q³,
   p is 0, 1 or 2;

Q² is a difluoroboron beta-diketone of formula (V)

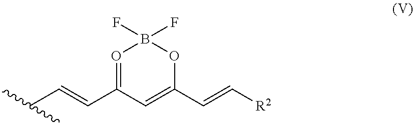

Q³ is a difluoroboron beta-diketone of formula (VI)

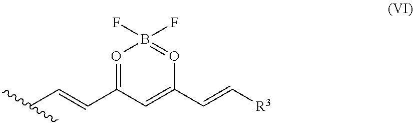

R¹, R² and R³, are each independently chosen among:

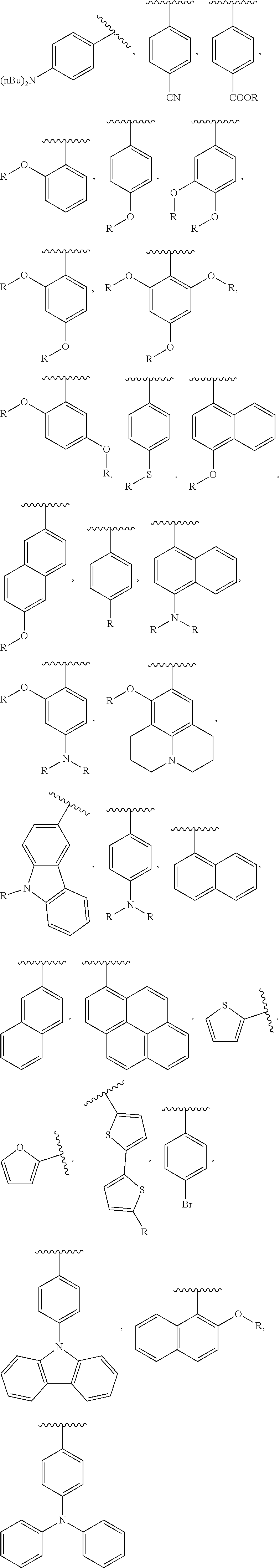

with R being each independently a hydrogen atom, a C₁-C₁₂ alkyl group, an C₆-C₁₀ aryl group, or a heteroaryl group, said alkyl, aryl and heteroaryl groups being optionally substituted.

Preferably, R¹, R² and R³, are each independently chosen:

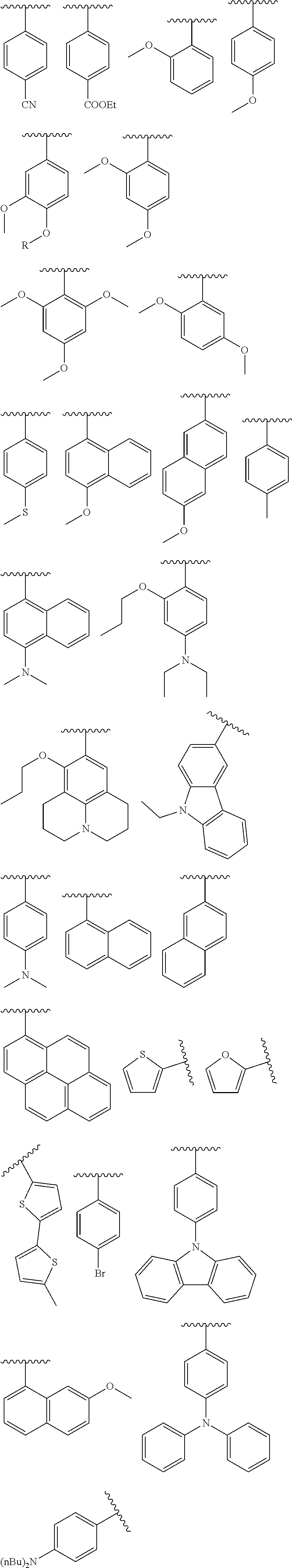

with R representing a methyl group.

The compounds of the invention exhibit enhanced optical properties in solution, such as high optical brightnesses obtained at one- and two-photon excitation. UV/visible absorption of solid-state particles comprising the compounds of the invention formed in water solution reveals that these compounds are strongly aggregated and fluorescence spectroscopy shows they are emissive in the NIR with enhanced fluorescence quantum yields in comparison with the compounds of prior art.

In the compounds of the invention, when Q¹ is represented by formula (II)

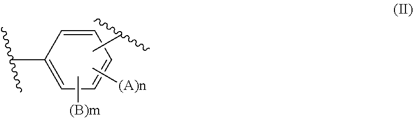

and n is 2, 3 or 4, the alkoxy groups A may be identical or different.

For the purposes of the present invention, the term “alkyl group” is understood to mean, an optionally substituted, saturated and linear, branched or cyclic carbon-comprising radical comprising from 1 to 12 carbon atoms, for example 1 to 8 carbon atoms. Mention may be made, as saturated and linear or branched alkyl, for example, of the methyl, ethyl, propyl, butyl, pentyl, hexyl, octyl, nonyl, decyl, undecyl and dodecanyl radicals and their branched isomers. Mention may be made, as cyclic alkyl, of the cyclopropyl, cyclobutyl, cyclopentyl, cyclohexyl, bicyclo[2.1.1]hexyl and bicyclo[2.2.1]heptyl radicals. The alkyl group, within the meaning of the invention, can optionally be substituted by one or more hydroxyl groups, one or more alkoxy groups, one or more halogen atoms chosen from the fluorine, chlorine, bromine and iodine atoms, one or more nitro (—NO₂) groups, one or more nitrile (—CN) groups or one or more aryl groups, with the alkoxy and aryl groups as defined in the context of the present invention.

The term “aryl” denotes generally an aromatic cyclic substituent comprising from 6 to 20 carbon atoms, for example 6 to 10. In the context of the invention, the aryl group can be mono- or polycyclic. Mention may be made, by way of indication, of the phenyl, benzyl and naphthyl groups. The aryl group can optionally be substituted by one or more hydroxyl groups, one or more alkoxy groups, one or more halogen atoms chosen from the fluorine, chlorine, bromine and iodine atoms, one or more nitro (—NO₂) groups, one or more nitrile (—CN) groups or one or more alkyl groups, with the alkoxy and alkyl groups as defined in the context of the present invention.

The term “heteroaryl” denotes generally an aromatic mono- or polycyclic substituent comprising from 5 to 10 members, including at least 2 carbon atoms, and at least one heteroatom chosen from nitrogen, oxygen, boron, silicon, phosphorus or sulfur. The heteroaryl group can be mono- or polycyclic. Mention may be made, by way of indication, of the furyl, benzofuranyl, pyrrolyl, indolyl, isoindolyl, azaindolyl, thiophenyl, benzothiophenyl, pyridyl, quinolinyl, isoquinolyl, imidazolyl, benzimidazolyl, pyrazolyl, oxazolyl, isoxazolyl, benzoxazolyl, thiazolyl, benzothiazolyl, isothiazolyl, pyridazinyl, pyrimidinyl, pyrazinyl, triazinyl, cinnolinyl, phthalazinyl and quinazobnyl groups. The heteroaryl group can optionally be substituted by one or more hydroxyl groups, one or more alkoxy groups, one or more halogen atoms chosen from the fluorine, chlorine, bromine and iodine atoms, one or more nitro (—NO₂) groups, one or more nitrile (—CN) groups, one or more aryl groups or one or more alkyl groups, with the alkyl, alkoxy and aryl groups as defined in the context of the present invention.

The term “alkoxy group” means an alkyl group as defined above, bonded via an oxygen atom (—O-alkyl).

The term “halogen atom” is understood to mean an atom chosen from the fluorine, chlorine, bromine or iodine atoms.

In the compounds of formula (I), when Q₁ is

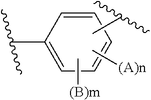

Q₂ and Q₃ may be in ortho, meta or para position.

According to a first embodiment of the invention, the compound of general formula (I) is a compound as defined above, wherein:

Q¹ is represented by formula (IIa)

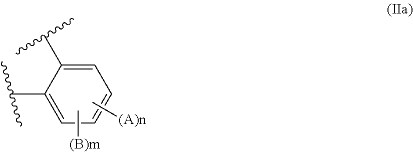

wherein
   Q¹ is substituted with Q² and Q³ in ortho position,
   A is a C₁-C₁₂ alkoxy group,
   B is a difluoroboron beta-diketone of formula (III),

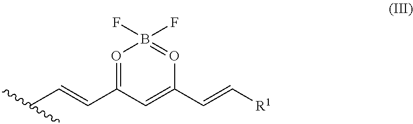

n is 0, 1, 2, 3 or 4,
m is 0 or 1; and

Q² is a difluoroboron beta-diketone of formula (V)

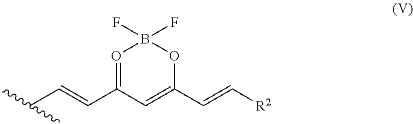

Q³ is a difluoroboron beta-diketone of formula (VI)

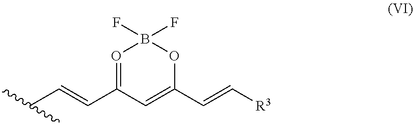

R¹, R² and R³, are each independently chosen among:

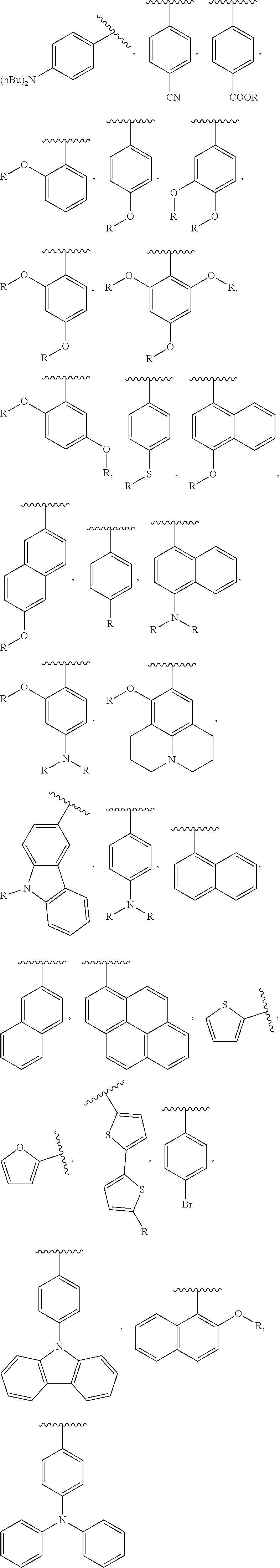

with R being each independently a hydrogen atom, a C₁-C₁₂ alkyl group, an C₆-C₁₀ aryl group, or a heteroaryl group, said alkyl, aryl and heteroaryl groups being optionally substituted.

According to a second embodiment of the invention, the compound of general formula (I) is a compound as defined above, wherein

Q¹ is represented by formula (IIa)

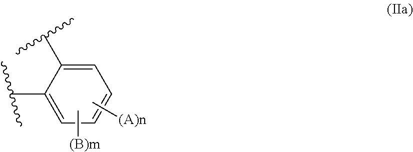

wherein
   Q¹ is substituted with Q² and Q³ in ortho position,
   n=m=0; and

Q² is a difluoroboron beta-diketone of formula (V)

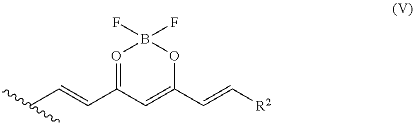

Q³ is a difluoroboron beta-diketone of formula (VI)

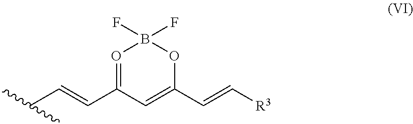

with R² and R³ being each independently chosen among:

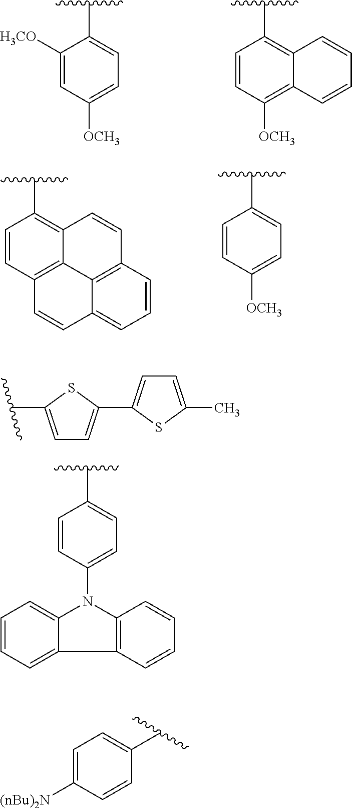

Examples of the first and second embodiments of the invention, are compounds 1 and 2:

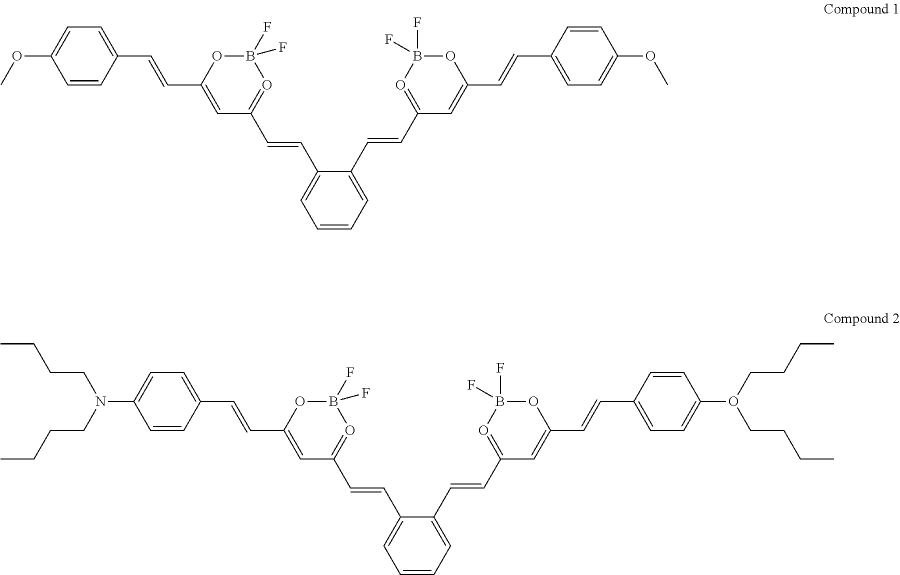

According to a third embodiment of the present invention, the compound of general formula (I) is a compound as defined above, wherein:

Q¹ is represented by formula (IIb)

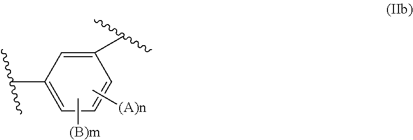

wherein
   Q¹ is substituted with Q² and Q³ in meta position,
   A is a C₁-C₁₂ alkoxy group,
   B is a difluoroboron beta-diketone of formula (III)

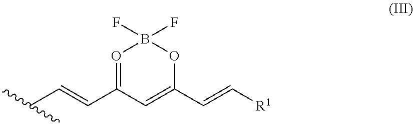

n is 0, 1, 2, 3 or 4,
m is 0 or 1; and

Q² is a difluoroboron beta-diketone of formula (V)

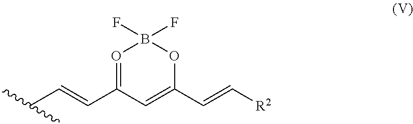

Q³ is a difluoroboron beta-diketone of formula (VI)

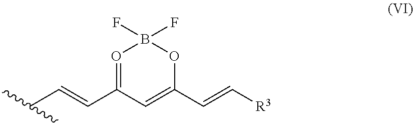

R¹, R² and R³, are each independently chosen among:

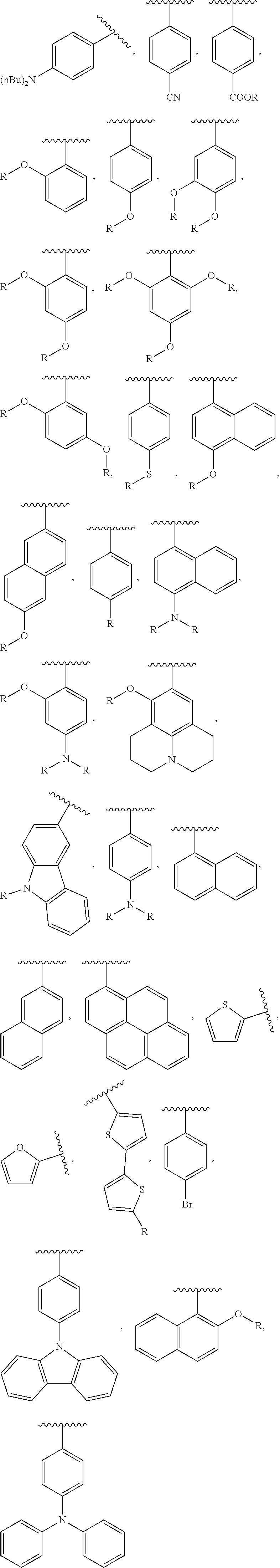

with R being each independently a hydrogen atom, a C₁-C₁₂ alkyl group, an C₆-C₁₀ aryl group, or a heteroaryl group, said alkyl, aryl and heteroaryl groups being optionally substituted.

According to a fourth embodiment of the present invention, the compound of general formula (I) is a compound as defined above, wherein:

Q¹ is represented by formula (IIb)

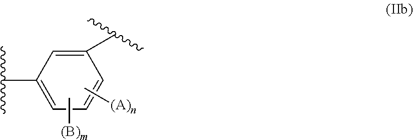

wherein
   Q¹ is substituted with Q² and Q³ in meta position,
   A is a C₁-C₁₂ alkoxy group,
   B is a difluoroboron beta-diketone of formula (III)

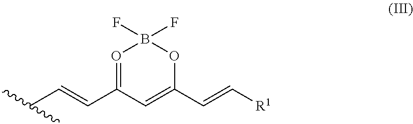

n is 0, 1, 2, 3 or 4,
m is 0 or 1; and

Q² is a difluoroboron beta-diketone of formula (V)

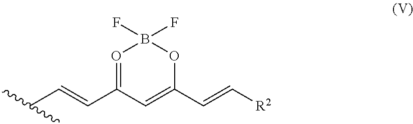

Q³ is a difluoroboron beta-diketone of formula (VI)

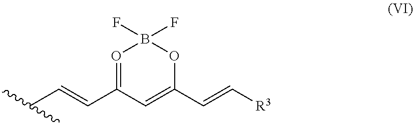

with R¹, R² and R³ being each independently chosen among:

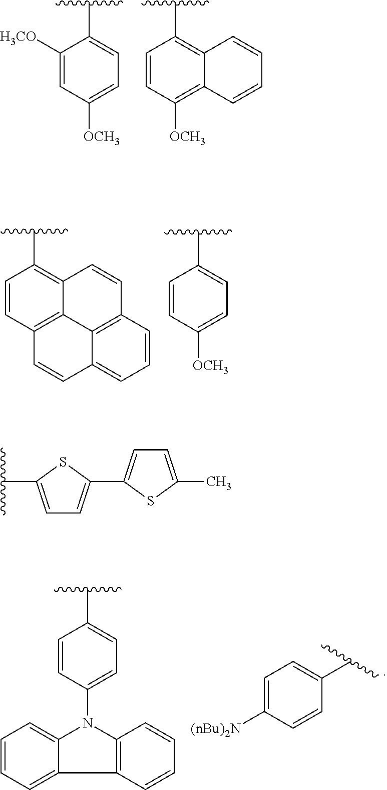

An example of the third and the fourth embodiments of the invention, is compound 3:

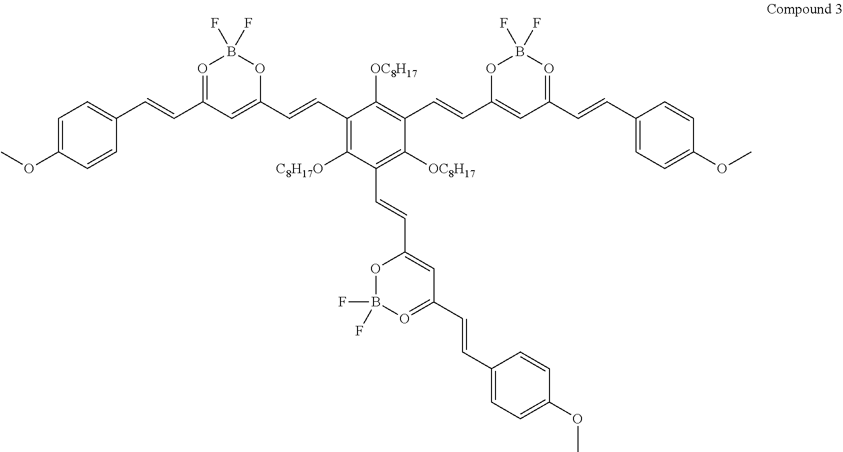

According to a fifth embodiment of the present invention, the compound of general formula (I) is a compound as defined above, wherein:

Q¹ is represented by formula (IIb)

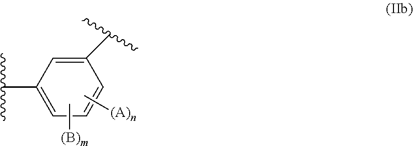

wherein
   Q¹ is substituted with Q² and Q³ in meta position,
   n=1, 2, 3 or 4,
   m=0,
   A is a C₁-C₁₂ alkoxy group; and

Q² is a difluoroboron beta-diketone of formula (V)

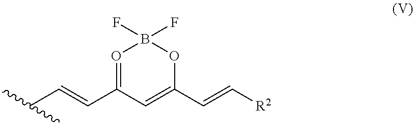

Q³ is a difluoroboron beta-diketone of formula (VI)

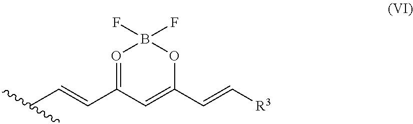

with R² and R³ being each independently chosen among:

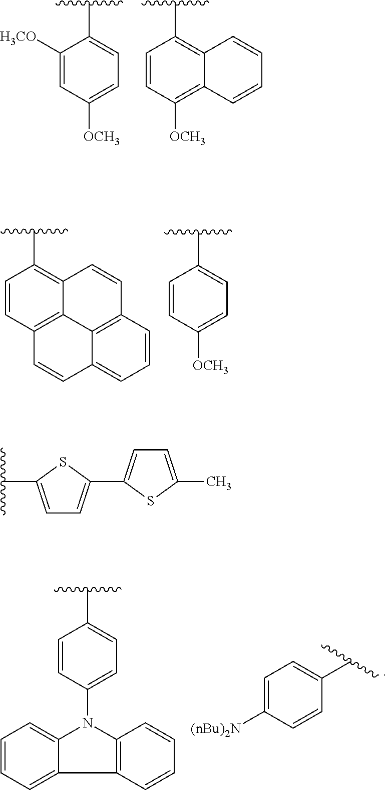

In this fifth embodiment, n is preferably equal to 3.

Examples of this fifth embodiment are compounds 4-12:

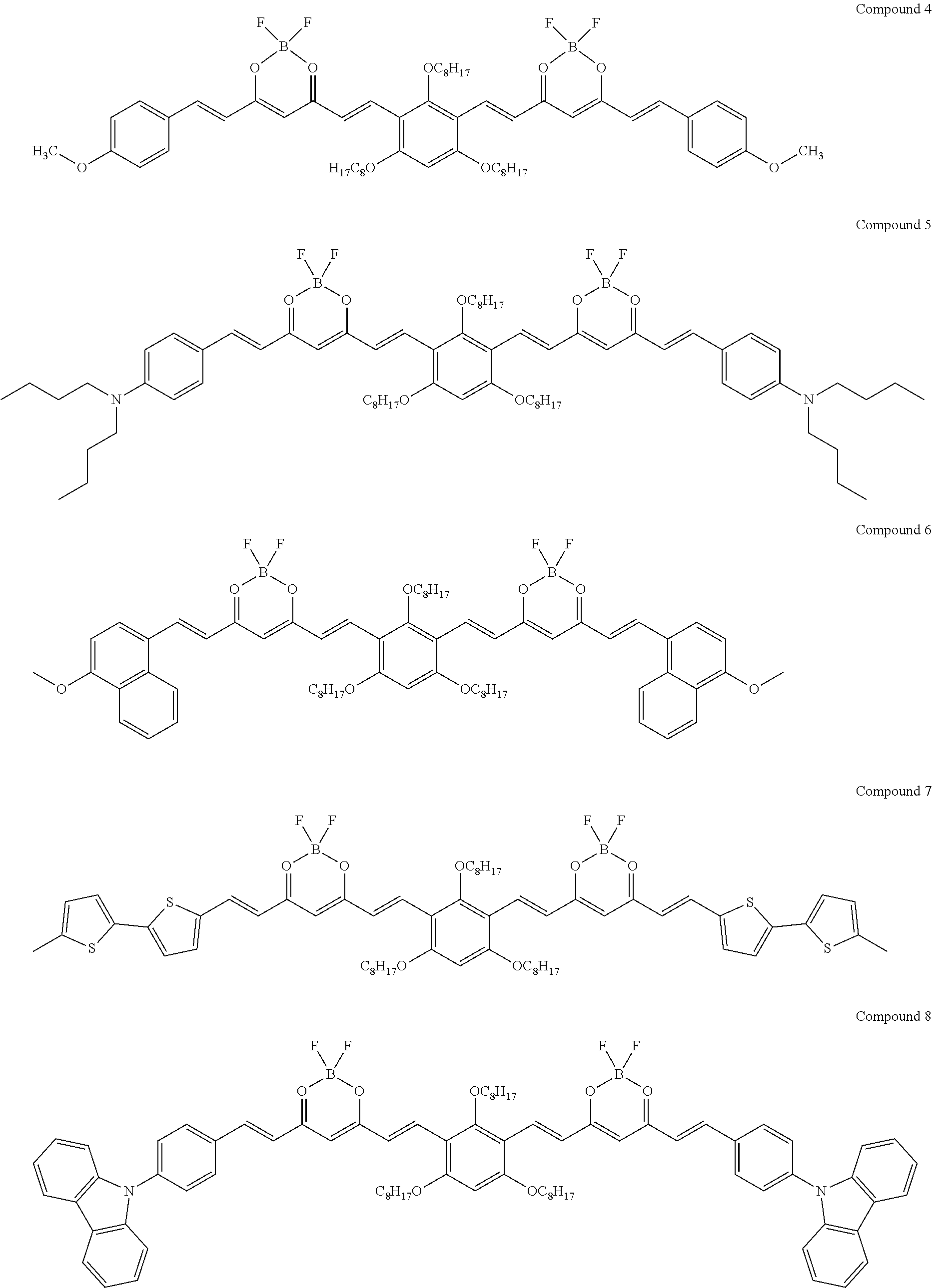

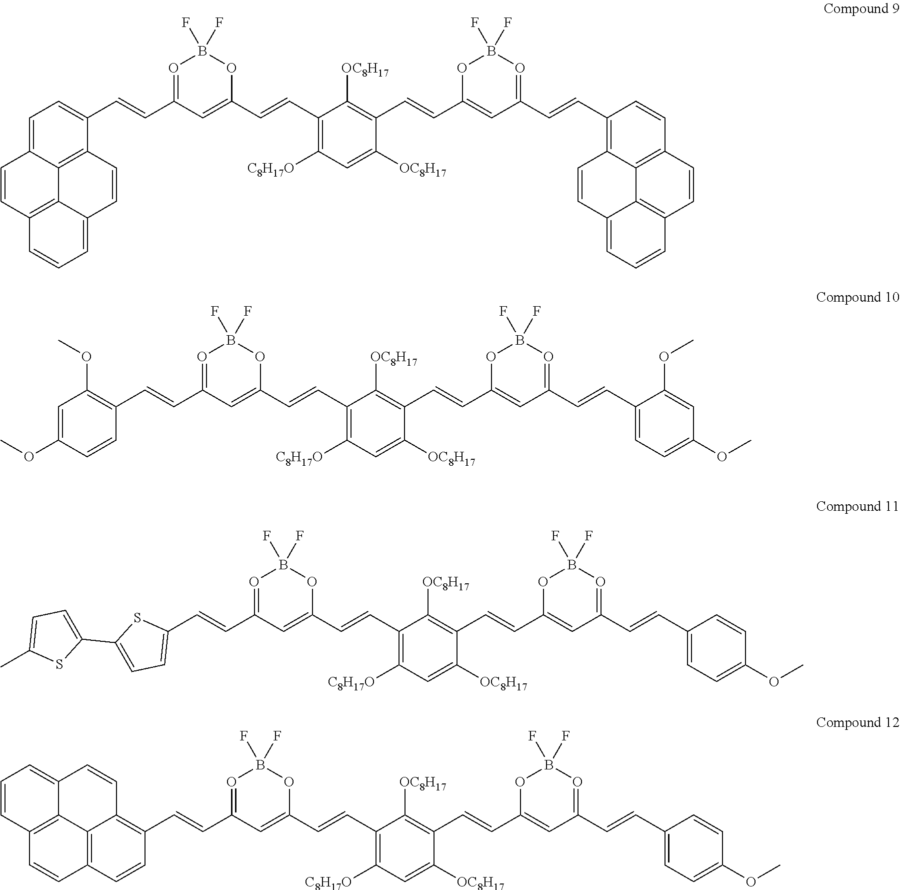

According to a sixth embodiment of the invention, the compound of general formula (I) is a compound as defined above, wherein:

Q¹ is represented by formula (IIc)

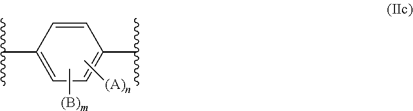

wherein
   Q¹ is substituted with Q² and Q³ in para position,
   A is a C₁-C₁₂ alkoxy group,
   B is a difluoroboron beta-diketone of formula (III)

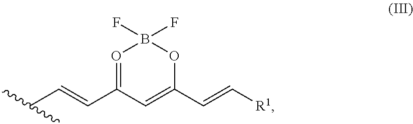

n is 0, 1, 2, 3 or 4,
m is 0 or 1; and

Q² is a difluoroboron beta-diketone of formula (V)

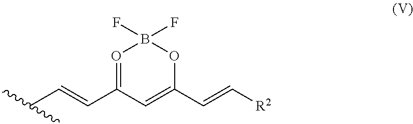

Q³ is a difluoroboron beta-diketone of formula (VI)

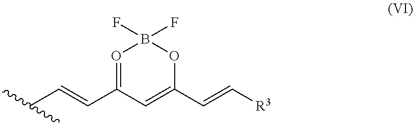

R¹, R² and R³, are each independently chosen among:

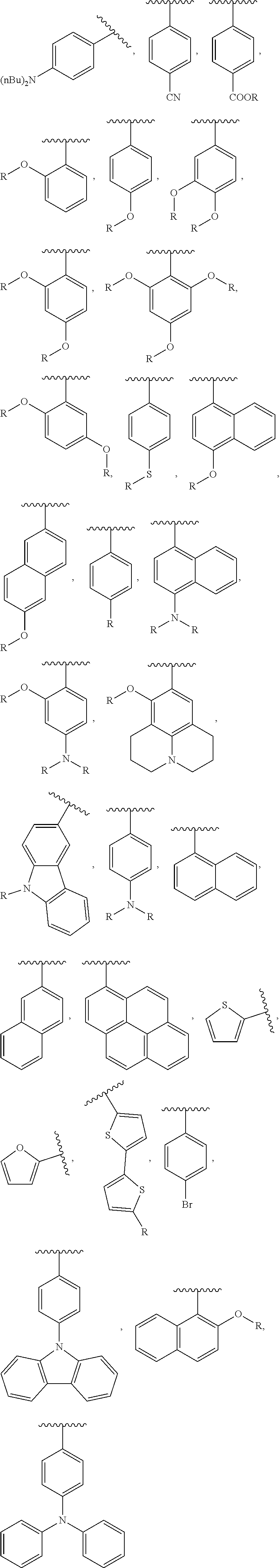

with R being each independently a hydrogen atom, a COCO alkyl group, an COCO aryl group, or a heteroaryl group, said alkyl, aryl and heteroaryl groups being optionally substituted.

According to a seventh embodiment of the invention, the compound of general formula (I) as defined above is a compound, wherein

Q¹ is represented by formula (IIc)

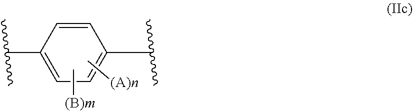

wherein
   Q¹ is substituted with Q² and Q³ in para position,
   A is a C₁-C₁₂ alkoxy group,
   B is a difluoroboron beta-diketone of formula (III),

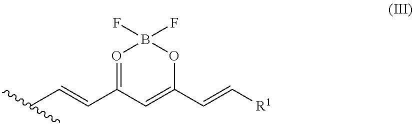

n is 0, 1, 2, 3 or 4.
m is 0 or 1;

Q² is a difluoroboron beta-diketone of formula (V)

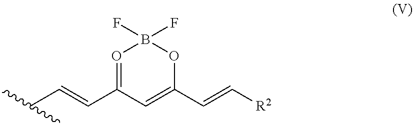

Q³ is a difluoroboron beta-diketone of formula (VI)

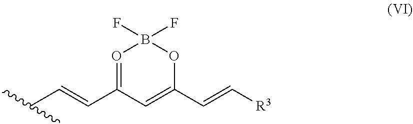

with R¹, R², and R³ being each independently chosen among:

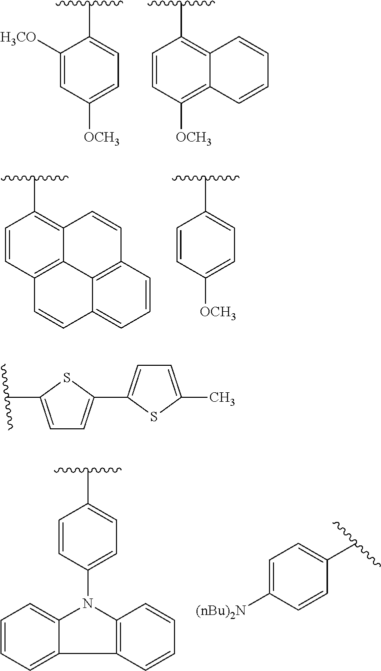

According to an eighth embodiment of the invention, the compound of general formula (I) as defined above is a compound, wherein:

Q¹ is represented by formula (IIc)

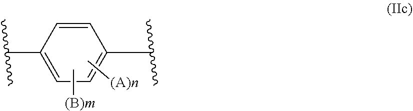

wherein
   Q¹ is substituted with Q² and Q³ in para position,
   A is a C₁-C₁₂ alkoxy group,
   n is 0, 1, 2, 3 or 4,
   m=0; and

Q² is a difluoroboron beta-diketone of formula (V)

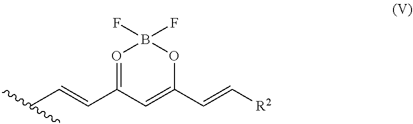

Q³ is a difluoroboron beta-diketone of formula (VI)

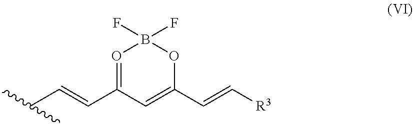

with R² and R³ being each independently chosen among:

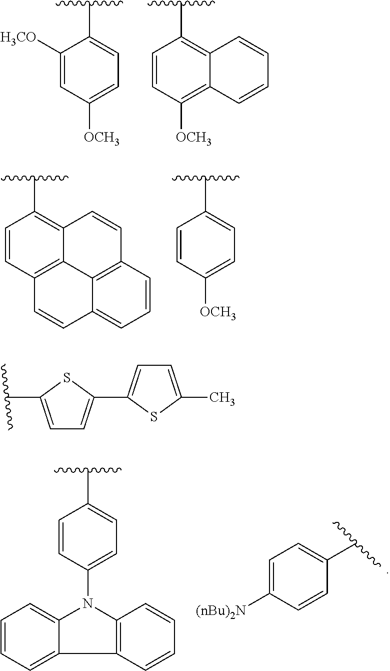

Examples of the sixth, seventh and the eighth embodiments of the invention are compounds 14-16:

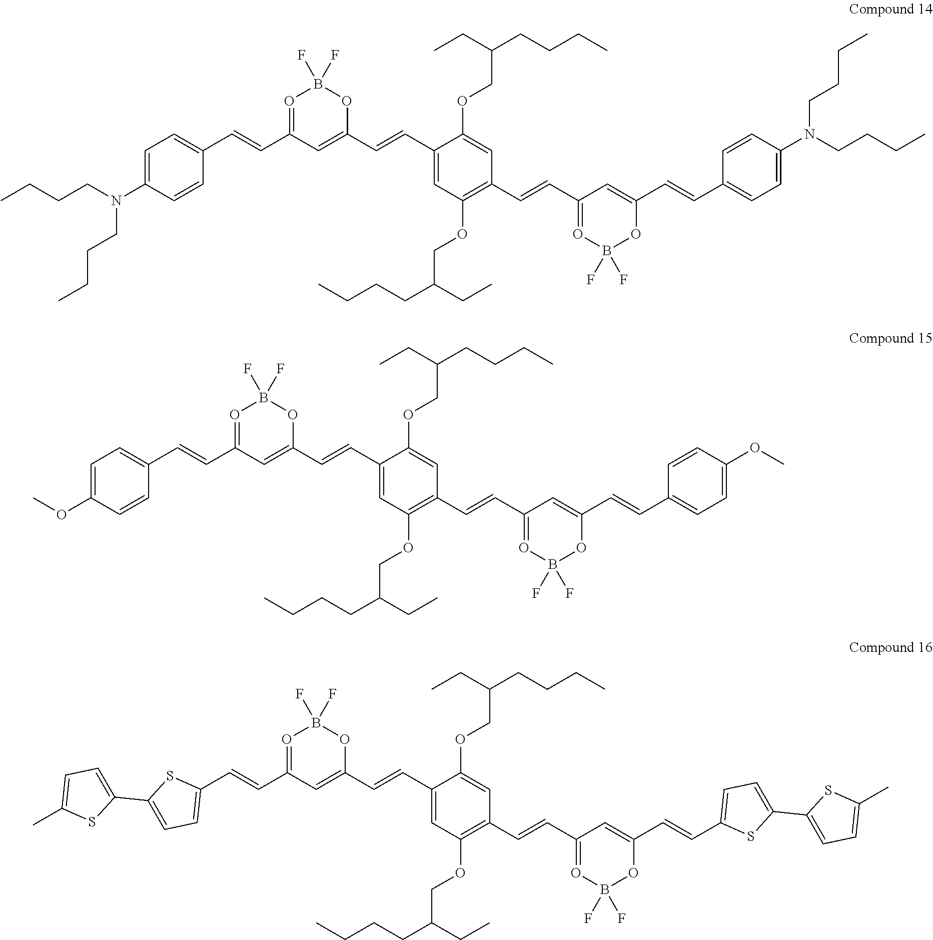

In embodiments one to eight, A is preferably a C₁-C₈ alkoxy.

According to a ninth embodiment of the present invention, the compound of general formula (I) as defined above is a compound, wherein:

Q¹ is represented by formula (IV)

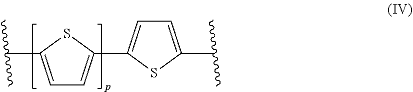

wherein
   Q¹ is substituted with Q² and Q³,
   p is 0, 1 or 2; and

Q² is a difluoroboron beta-diketone of formula (V)

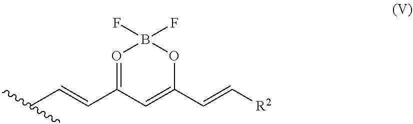

Q³ is a difluoroboron beta-diketone of formula (VI)

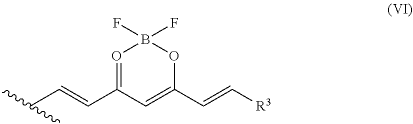

R¹, R² and R³, are each independently chosen among:

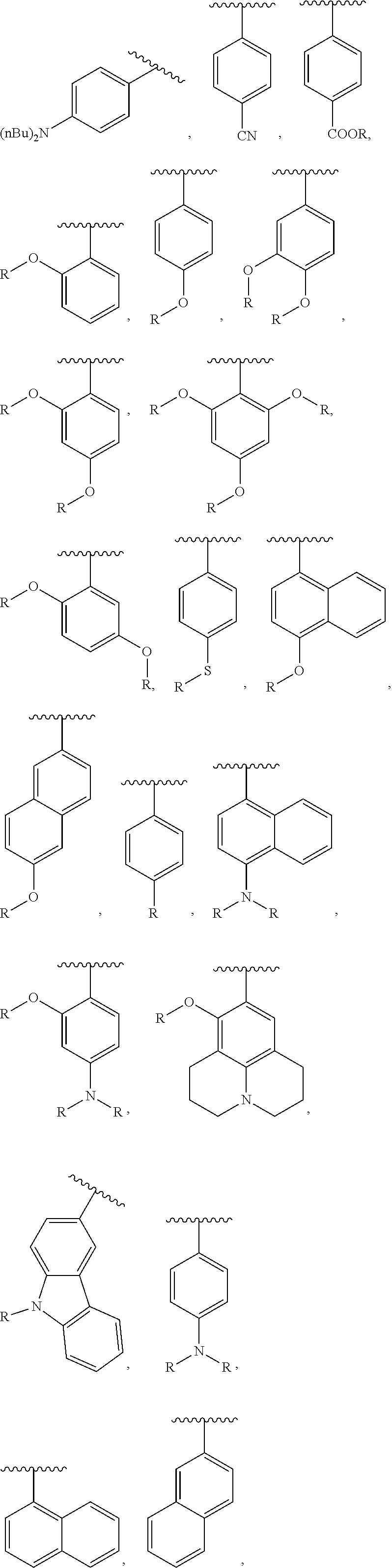

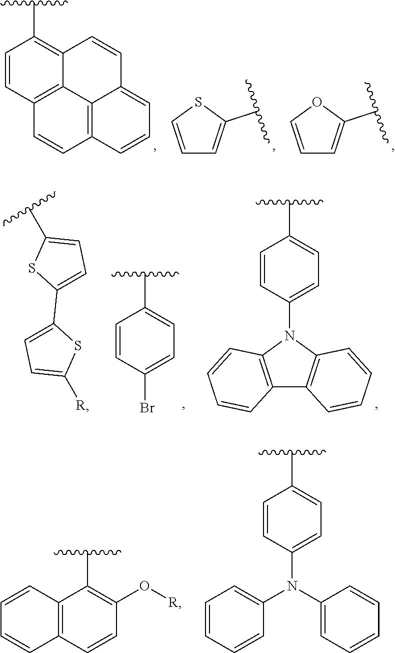

with R being each independently a hydrogen atom, a C₁-C₁₂ alkyl group, an C₆-C₁₀ aryl group, or a heteroaryl group, said alkyl, aryl and heteroaryl groups being optionally substituted.

According to a tenth embodiment of the invention, the compound of general formula (I) as defined above is a compound, wherein:

Q¹ is represented by formula (IV)

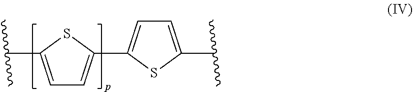

wherein
   Q¹ is substituted with Q² and Q³,
   p is 0, 1 or 2; and

Q² is a difluoroboron beta-diketone of formula (V)

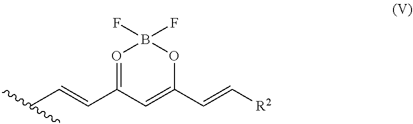

Q³ is a difluoroboron beta-diketone of formula (VI)

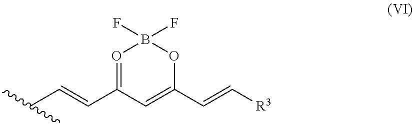

with R² and R³ being each independently chosen among:

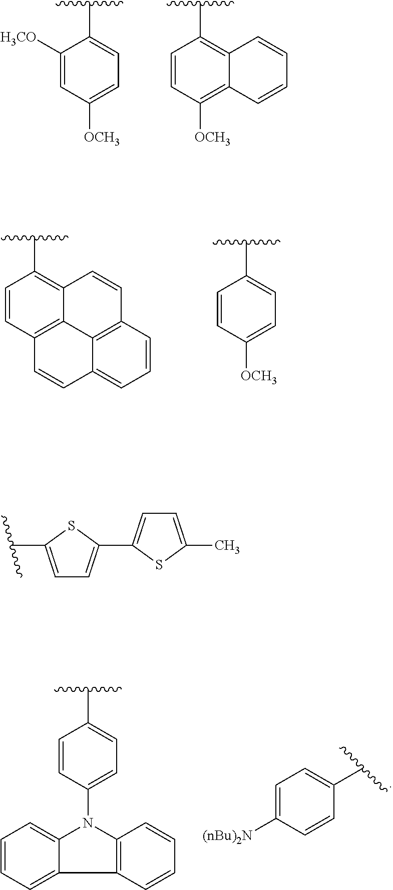

Examples of the ninth and tenth embodiments of the invention are compounds 17-21:

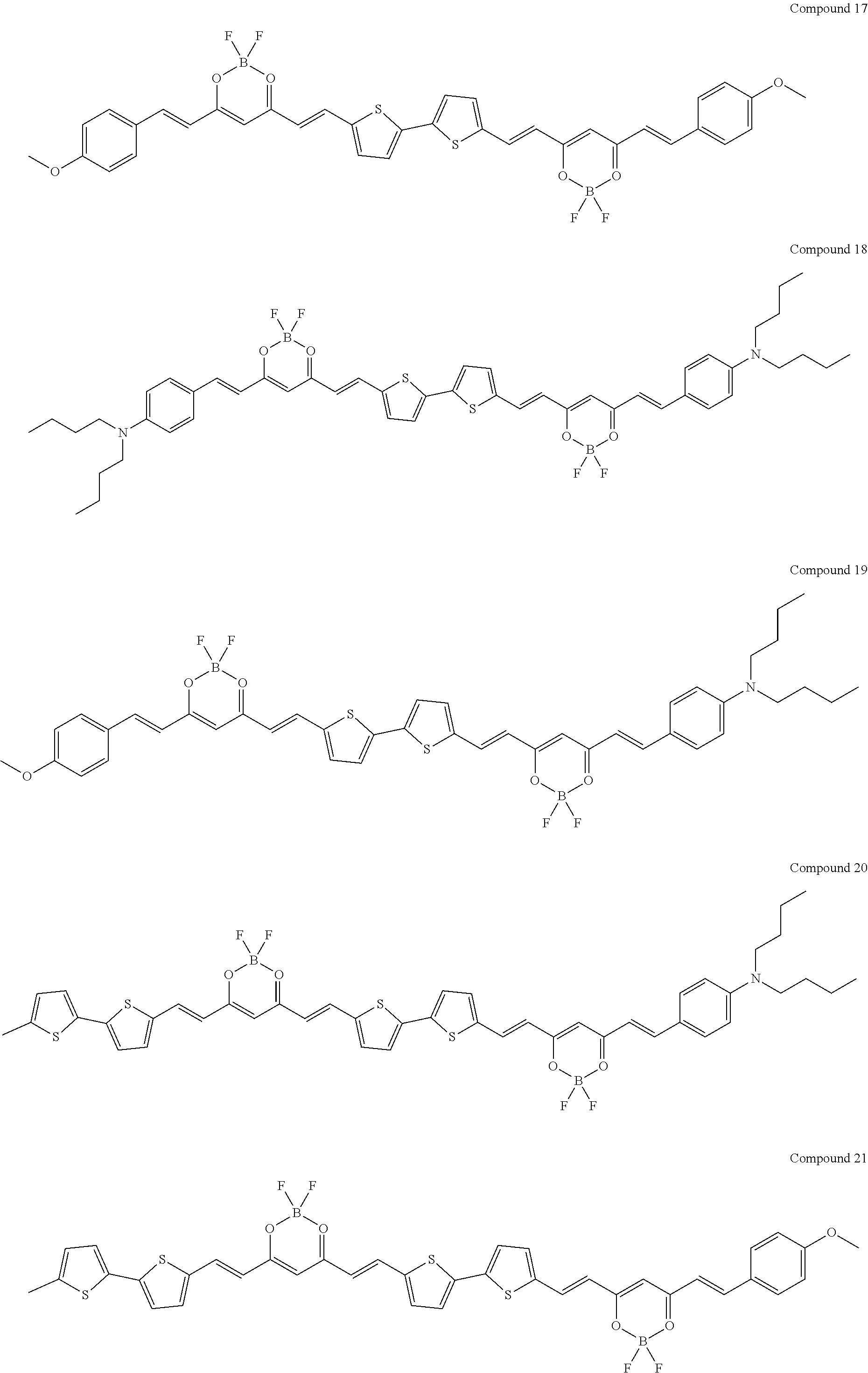

In the first, third, sixth, ninth embodiments, R¹, R² and R³ are preferably independently chosen among:

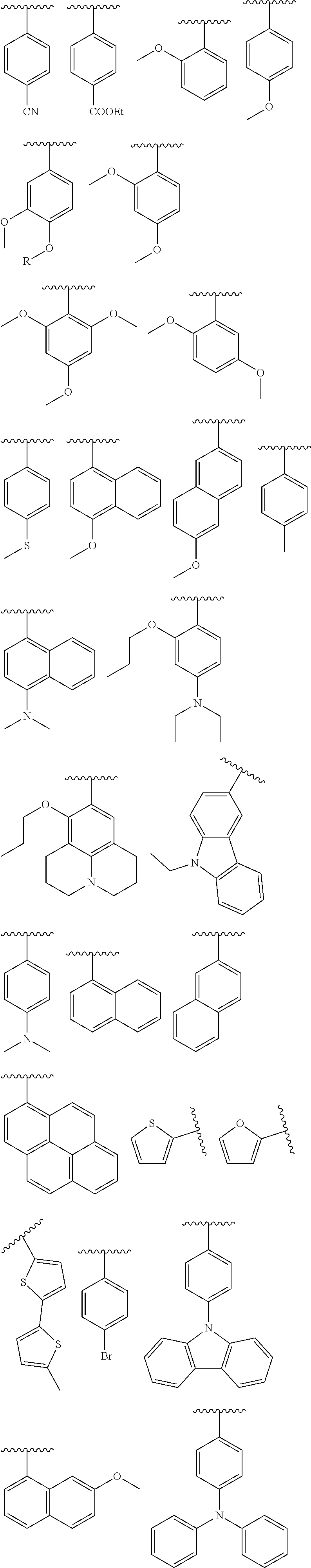

with R representing a methyl group.

In all embodiments, when R is an alkyl group, R is preferably a linear, non-substituted alkyl group.

The compounds of the invention can be obtained according to the protocol described in G. Mann, L. Beyer and A. Arrieta, Z. Chem., 1987, 27, 172-173 or in J. Med Chem. 2006, 49, 6111-6119, as shown below:

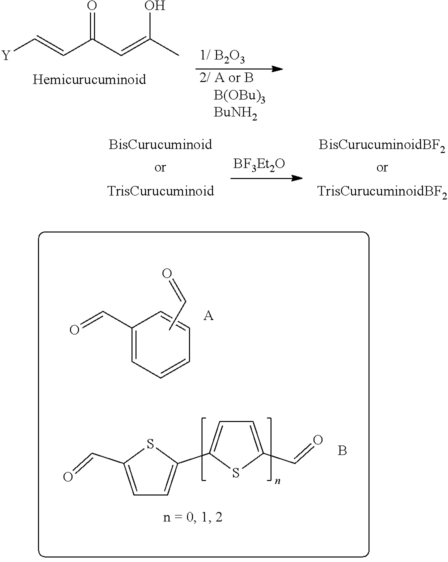

with Y chosen among:

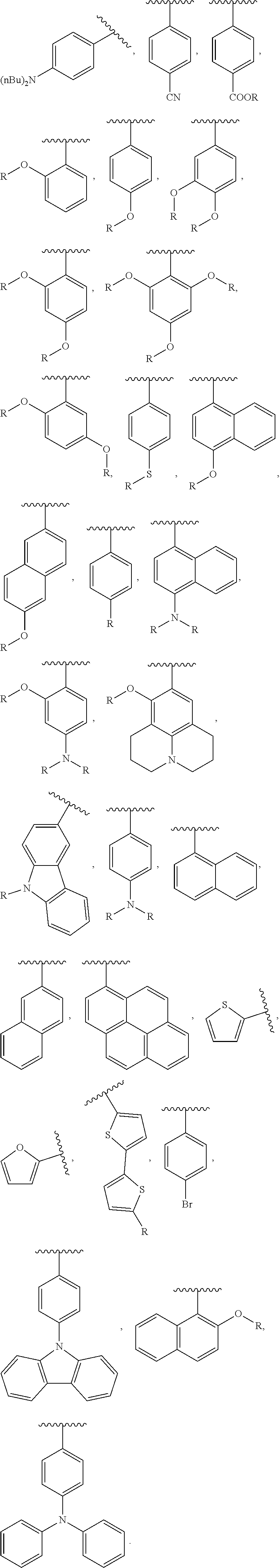

### Synthesis of Curcuminoid

In a round bottom flask, a solution of hemicurcuminoid (1 mol eq) and B₂O₃ (0.5 mol eq) dissolved in ethyl acetate (DMF) (10 mL) was stirred at 60° C. for 30 min. A solution of the appropriate bis-aldehyde (A or B) or tris-aldehyde (A) (1 mol eq of aldehyde function) and tri(n-butyl)borane (1 mol eq of aldehyde function) in ethyl acetate (10 mL) was added and the mixture was stirred for 30 min at 60° C. A catalytic amount of n-butylamine (0.5 mol eq) was then added to the solution and the reaction mixture was refluxed overnight. After cooling to 60° C. 30 mL of 0.4 M HCl were added and the mixture was stirred for 30 min. After cooling, the precipitate was filtered off and dried in vacuo to yield the pure BisCurcuminoid or TrisCurcuminoid-. When the solid was not pure, it was purified by column chromatography using silica gel as stationary phase.

### Synthesis of CurcuminoidBF₂

In a round bottom flask, the ligand (1 mol eq) was solubilized in dichloromethane (20 mL) before boron trifluoride etherate (1.1 mol eq) was added. The reaction mixture was refluxed overnight. After cooling to room temperature, the solvent was evaporated and the resulting solid was suspended in diethyl ether. The precipitate was filtered off yielding the pure complex. When the crude complex was not pure, it was purified by column chromatography using silica gel as stationary phase.

The inventors have shown that compounds of the invention, display a high fluorescence quantum yield (12.5% in water with maximum emission at 692 nm and 61% in dichloromethane with maximum emission at 574 nm), and a good value of the two-photon absorption cross section (560 GM in water with excitation at 850 nm and 510 GM in dichloromethane with excitation at 800 nm), which makes them attractive fluorophores. These fluorophores have improved NIR emitting properties particularly useful in the solid state for imaging applications, for example.

Another aspect of the invention concerns the use of the compounds of formula (I) as a fluorophore. The compounds of the invention can be used both in solution, in particular in organic solvents, and in solid-state.

The present invention also concerns the use of the compounds of formula (I) in bioimaging, in particular for cells imaging; as sensors of volatile acid/base; in photodynamic therapy; in diagnosis of Alzheimer's disease; in theranostics; as optical sensors for anaerobic environment; in display and telecommunication technologies, in photovoltaics.

The compounds of the invention can represent fluorescent reporters for human beta-amyloid peptide, produced in the nerve tissues and in the blood in the course of Alzheimer's disease and may thus be used in diagnosis of Alzheimer's disease.

In the photovoltaics, the compounds of the invention may be considered as electron donors in solar cells.

Other advantages and features of the present invention may be better understood with respect to the following examples given for illustrative purposes and the accompanying figures.

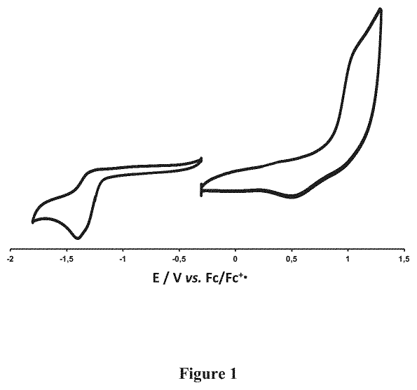

FIG. 1 represents the cyclic voltammogram of compound 4 in dichloromethane (DCM) solution containing 0.1M [(ⁿBu₄N)PF₆] (Scan rate of 100 mV/s).

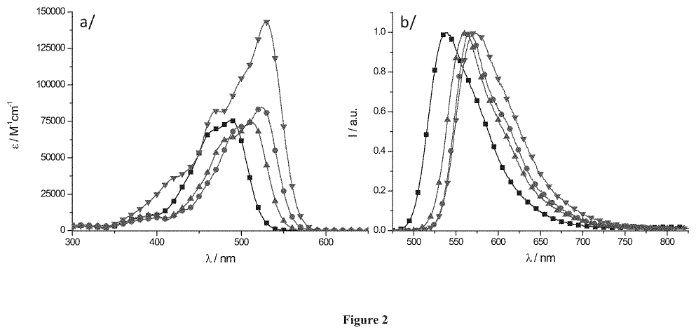

FIG. 2 a/ Electronic absorption spectra and corrected normalized fluorescence emission spectra (cone. ≈10⁻⁶ M. λ_{exc} at the absorption maximum) of compounds X (-, ▪), Y ( [symbol] , [symbol] ), Z ( [symbol] , [symbol] ) and 4 ( [symbol] , [symbol] ) recorded in DCM at room temperature.

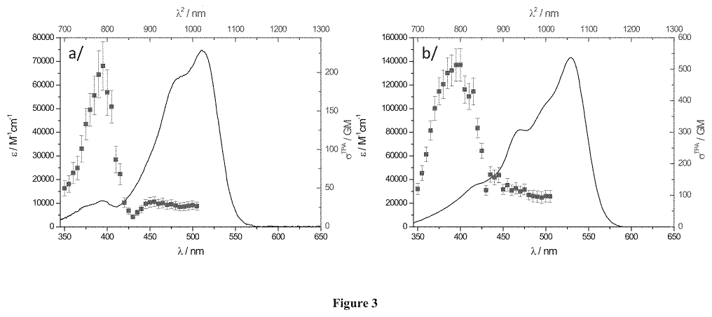

FIG. 3 represents the two-photon excitation ( [symbol] , higher x-coordinate and [symbol] , right y-coordinate) with their error bars, ortho-phtalaldehyde (OPA) spectra (-, lower x-coordinate and -, left y-coordinate) in DCM: a/ compound Z and b/ compound 4.

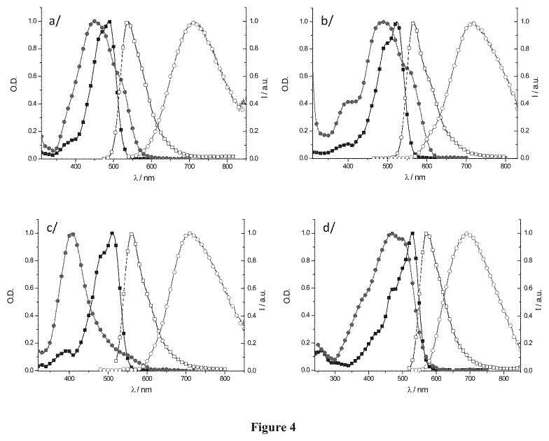

FIG. 4 represents the UV/visible absorption spectra of DCM solutions (-, ▪) and particles in water ( [symbol] ) and fluorescence spectra of DCM solutions (- - -, □) and particles in water ( [symbol] ) for a/ compound X; b/ compound Y; c/ compound Z and d/ compound 4.

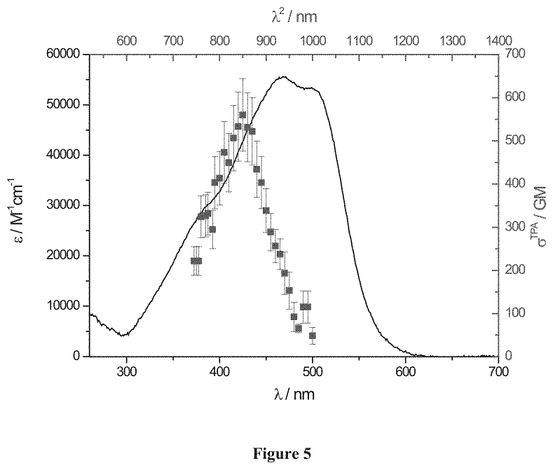

FIG. 5 represents the two-photon excitation ( [symbol] , higher x-coordinate and [symbol] , right y-coordinate) with their error bars, OPA spectra (-, lower x-coordinate and -, left y-coordinate) of particles of compound 4 in water.

### EXAMPLES

### Materials and Methods

### Spectroscopy Measurements

Spectroscopy measurements were carried out with spectroscopic grade solvents. NMR spectra (¹H. ¹³C. ¹⁹F) were recorded at room temperature on a BRUKER AC 250 operating at 400, 100, and 425 MHz for ¹H, ¹³C, and ¹⁹F, respectively. Data are listed in parts per million (ppm) and are reported relative to tetramethylsilane (¹H and ¹³C); residual solvent peaks of the deuterated solvents were used as internal standards. Mass spectra were realized in Spectropole de Marseille (http://www.spectropols.fr/). Solid state spectra and luminescence quantum yield were measured using an integrating sphere. UV/Vis-absorption spectra were measured on a Varian Cary 50. Emission spectra were measured on a Horiba-JobinYvon Fluorolog-3 spectrofluorimeter that was equipped with a three-slit double-grating excitation and a spectrograph emission monochromator with dispersions of 2.1 nm·mm⁻¹ (1200 grooves·mm⁻¹). Steady-state luminescence excitation was done using unpolarized light from a 450W xenon CW lamp and detected at an angle of 90° for dilute-solution measurements (10 mm quartz cell) and with a red-sensitive Hamamatsu R928 photomultiplier tube. Special care was taken to correct NIR-emission spectra that were obtained with the latter device. The detector was corrected according to the procedure described by Parker et al. (C. A. Parker, in Photoluminescence of Solutions, Elsevier Publishing, Amsterdam, 1969). The observed photomultiplier output A₁ was recorded at a wavelength λ, which corresponds to the apparent emission spectrum. A₁ is given by [Eq. (1)], where F₁ and S₁ are the corrected emission spectrum and the spectroscopic sensitivity factor of the monochromator-photomultiplier setup, respectively.

A₁=(F₁)(S₁)/λ² (Eq. 1)

To calculate S₁, T-N,N-dimechylamino-4′-nitrostilbene (DMANS) is used as a standard NIR fluorophore for which its corrected emission spectrum has been precisely determined (J. R. Lakowicz, Principles of Fluorescence Spectroscopy, 3rd edn., Kluwer, New-York (NY), 2006). Luminescence quantum yields (Φ_{f}) were measured in dilute DCM solutions with an absorbance below 0.1 by using [Eq. (2)], where OD(λ) is the absorbance at the excitation wavelength (λ), n the refractive index, and I the integrated luminescence intensity.

Φ_{fx}/Φ_{fr}=[ODᵣ(λ)/ODₓ(λ)][Iₓ/Iᵣ][nₓ/nᵣ] (Eq. 2)

Subscripts “r” and “x” stand for reference and sample, respectively. The luminescence quantum yields were not corrected by the refractive indices. We used ruthenium trisbipyridine bischloride in water (Φ_{fr}=0.021) as a reference for compounds that absorbed in the 450 nm region, while rhodamine B (Φ_{fr}=0.49) in EtOH was used for excitation between 540 and 560 nm.

Lifetime measurements were carried out on a HORIBA Jobin Yvon IBH FluoroLog-3 spectrofluorimeter that was adapted for time-correlated single-photon counting. For these measurements, pulsed LEDs with an appropriate wavelength were used. Emission was monitored perpendicular to the excitation pulse and spectroscopic selection was achieved by a passage through the spectrograph. A thermoelectrically cooled single-photon-detection module (HORIBA Jobin Yvon IBH, TBX-04-D) incorporating a fast-rise-time photomultiplier tube, a wide-bandwidth preamplifier, and a picosecond-constant fraction discriminator was used as the detector. Signals were acquired using an IBH DataStation Hub photon counting module and data analyses were performed by using the commercially available DAS 6 decay-analysis software package from HORIBA Jobin Yvon IBH; the reported τ values are given with an estimated uncertainty of about 10%.

Cyclic voltammetric (CV) data were acquired using a BAS 100 Potentiostat (Bioanalytical Systems) and a PC computer containing BAS100W software (v2.3). A three-electrode system with a Pt working electrode (diameter 1.6 mm), a Pt counter electrode and an Ag/AgCl (with 3M NaCl filling solution) reference electrode was used. [(nBu)₄N]PF₆ (0.1 M in dichloromethane) served as an inert electrolyte. Cyclic voltammograms were recorded at a scan rate of 100 mV·s⁻¹. Ferrocene was used as internal standard (N. G. Connelly and W. E. Geiger, Chem. Rev., 1996, 96, 877-910).

### Example 1: Synthesis of Compound 4

The synthesis of compound 4 is reported below. The two-photon excited fluorescence (TPEF) properties of 4 are characterized in solution. The preparation and fluorescence emission of organic nanoparticles obtained using 4 are also described. This approach allows giving an example of a compound as defined in the invention with improved emission ability in both the solution and the solid state.

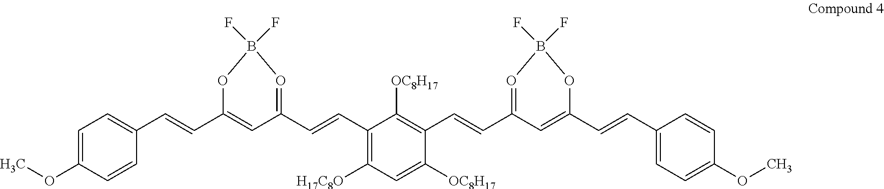

Synthesis of compound 4 requires first of all the preparation of compound A. This intermediate is prepared by a Knoevenagel reaction using an excess of acetylacetone (acac/aldehyde 3:1), providing compound A in a reasonable yield of 60% (G. Mann, L. Beyer and A. Arrieta, Z. Chem. 1987, 27, 172-173). Then, the reaction of two equivalents of A with 1,3,5-tris(n-octyloxy)benzene afforded 4 in a yield of 34%.

Complexation to boron difluoride is performed by reacting compound B with a slight excess of the boron trifluoride etherate in dichloromethane solution (DCM). Compound 4 is purified by crystallization or, when necessary, by column chromatography. Compound 4 is obtained as highly colored solids and characterized by ¹H- and ¹⁹F-NMR spectroscopies and by high resolution mass spectrometry (HRMS).

### Synthesis of compound A (1E,4Z)-5-hydroxy-1-(4-methoxyphenyl)hexa-1,4-dien-3-one)

In a 50 mL round bottom flask, a solution of acetylacetone (3.00 g, 30 mmol) and B₂O₃ (1.050 g, 15 mmol) dissolved in ethyl acetate (10 mL) was stirred at 60° C. for 30 min. A solution of anisaldehyde (1.36 g, 10 mmol) and tri(n-butyl)borane (2.30 g, 10 mmol) in ethyl acetate (8 mL) was added and the mixture was stirred for 30 min at 60° C. A catalytic amount of n-butylaminc (0.44 g, 6 mmol) was then added to the solution and the reaction mixture was refluxed overnight. After cooling to 60° C., 30 mL of 0.4 M HCl were added and the mixture was stirred for 30 min. After cooling, the precipitate was filtered off. The residual solution was evaporated. The oily compound was dissolved in dichloromethane. The organic layer was washed with water, brine, dried over MgSO₄ and evaporated to dryness. The oily crude was purified by column chromatography on silica using a mixture of cyclohexane and dichloromethane (gradient from 1/1 to 3/1) yielding the pure A as a yellowish solid (1.20 g, 55%).

### Synthesis of compound B ((1E,1′E,4Z,4′Z,6E,6′E)-1,1′-(2,4,6-tris(octyloxy)-1,3-phenylene)bis(5-hydroxy-7-(4-methoxyphenyl)hepta-1,4,6-trien-3-one))

In a 50 mL round bottom flask, a solution of A (1 mol eq) and B₂O₃ (0.5 mol eq) dissolved in ethyl acetate (10 mL) was stirred at 60° C. for 30 min. A solution of the appropriate aldehyde (1 mol eq of aldehyde function) and tri(n-butyl)borane (1 mol eq of aldehyde function) in ethyl acetate (10 mL) was added and the mixture was stirred for 30 min at 60° C. A catalytic amount of n-butylamine (0.5 mol eq) was then added to the solution and the reaction mixture was refluxed overnight. After cooling to 60° C., 30 mL of 0.4 M HCl were added and the mixture and stirred for 30 min. After cooling, the precipitate was filtered off and dried in vacuo to yield the pure compound B.

### Compound B: ((1E,1′E,4Z,4′Z,6E,6′E)-1,1′-(2,4,6-tris(octyloxy)-1,3-phenylene)bis(5-hydroxy-7-(4-methoxyphenyl)hepta-1,4,6-trien-3-one))

Dark orange solid; yield: 34%; ³H NMR (400 MHz, CDCl₃, ppm): δ=7.92 (d, ³J=16.1 Hz, 2H), 7.60 (d, ³J=15.8 Hz, 2H), 7.49 (d, ³J=8.7 Hz, 4H), 7.04 (d, ³J=16.1 Hz, 2H), 6.89 (d, ³J=8.7 Hz, 4H), 6.48 (d, ³J=15.8 Hz, 2H), 6.23 (s, 1H), 5.69 (s, 2H), 4.05 (t, ³J=6.2 Hz, 4H), 3.83 (m, 6H), 3.77 (t, ³J=6.3 Hz, 4H), 1.88 (m, 6H), 1.53 (m, 6H), 1.30 (m, 24H), 0.87 (m, 9H); ¹³C NMR (100 MHz, CDCl₃): 3=184.09, 183.78, 161.66, 161.23, 139.80, 131.86, 129.78, 128.00, 125.68, 122.29, 114.41, 111.44, 101.76, 92.63, 69.04, 55.47, 31.97, 29.68, 29.53, 29.44, 29.17, 26.42, 26.36, 22.81, 14.23. HRMS (ESI+) [M+2H]²⁺ calcd for C₅₈H₈₀O₈⁺ m/z=460.2901, found m/z=460.2903.

### General Procedure for Borondifluoride Complexes

In a 50 mL round bottom flask, the ligand (1 mol eq) was solubilized in dichloromethane (20 mL) before boron trifluoride etherate (1.1 mol eq) was added. The reaction mixture was refluxed overnight. After cooling to room temperature, the solvent was evaporated and the resulting solid was suspended in diethyl ether. The precipitate was filtered off yielding the pure compound 4.

### Compound 4: ((1E,1′E,4Z,4′Z,6E,6′E)-1,1′-(2,4,6-tris(octyloxy)-1,3-phenylene)bis(5-(difluoroboryloxy)-7-(4-methoxyphenyl)hepta-1,4,6-trien-3-one))

Red solid; yield: 97%; ³H NMR (400 MHz, DMSO, ppm): δ=8.25 (d, ³J=16 Hz, 2H), 7.97 (d, ³J=15.5 Hz, 2H), 7.55 (d, ³J=8.5 Hz, 4H), 7.14 (d, ³J=16 Hz, 2H), 6.93 (d, ³J=8.5 Hz, 4H), 6.54 (d, ³J=15.5 Hz, 2H), 6.26 (s, 1H), 5.56 (s, 1H), 4.14 (t, ³J=6.5 Hz, 4H), 3.85 (s, 6H), 3.80 (t, ³J=6.5 Hz, 2H), 1.92 (m, 6H), 1.32 (m, 30H), 0.90 ppm (m, 9H); ¹⁹F NMR (235 MHz, DMSO): δ=−140.91 (¹⁰B—F, 0.2), −140.97 ppm (¹¹B—F, 0.8). HRMS (ESI+) [M+2Na]²⁺ calcd for C₅₈H₇₆O₉B₂F₄Na₂²⁺ m/z=530.2707, found m/z=530.2705.

### Example 2: Electrochemical, Photophysical and Solid-Date Optical Properties of Compound 4 and Comparison with Other Compounds

It is reported herein a study of electrochemical and optical properties in organic solvents of four compounds: three borondifluoride complex of a mono-curcuminoid compounds (named X, Y and Z) versus compound 4, a borondifluoride complex of a bis-curcuminoid system. The two-photon excited fluorescence (TPEF) properties of the four compounds were characterized in solution.

Molecular structures of the borondifluoride curcuminoid derivatives X, Y, Z are presented below.

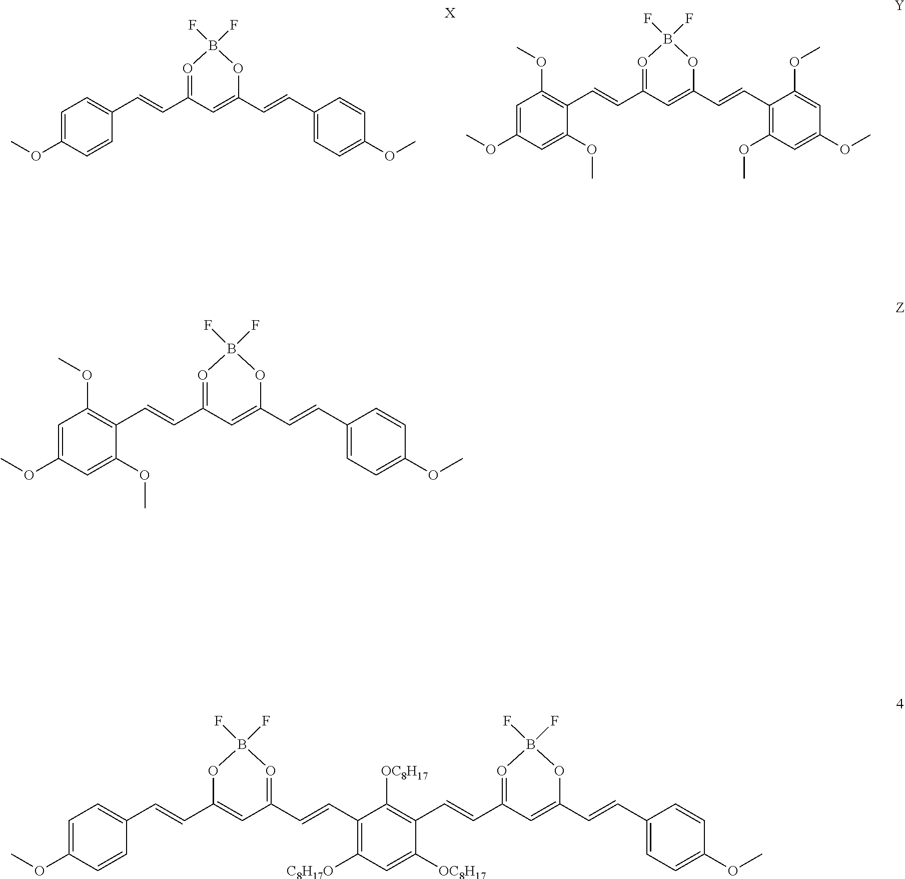

### 2.1. Comparison of Electrochemical Properties

The electrochemical properties of compounds X, Y, Z and 4 were investigated in dichloromethane (DCM) solution containing 0.1 M of (n-Bu)₄NPF₆.

The cyclic voltammograms (CV) are given in FIG. 1 and the oxidation and reduction half-wave potential values (E_{1/2} vs. ferrocene/ferrocenium) are collected in Table 1.

TABLE 1
Oxidation and reduction half-wave potential
values for the studied dioxaborine
derivatives (10 μM) in dichloromethane
solution vs. Fc/Fc+● using tetrabutylammonium
hexafluorophosphate as electrolyte (100 μM),
Electrochemical HOMO-LUMO gap
the values are given in volt. (a)
ΔEg = (E1/2ox)1 − E1/2red,
(b) The second oxidation process occurs at potentials
out of the solvent electrochemical window.

| Compound | E1/2red | (E1/2ox)1 | (E1/2ox)2 | ΔEg |
|---|---|---|---|---|
| X | −1.27 | 1.10 | −b | 2.37 |
| Y | −1.44 | 0.72 | 1.09 | 2.16 |
| Z | −1.37 | 0.89 | 1.31 | 2.26 |
| 4 | −1.30 | 1.05 | −b | 2.35 |

The increase of the oxidation potential in 4 relative to Z could be due to presence the second curcuminoid moiety. In addition, since both reduction and oxidation processes of 4 involve the same number of electrons, it may be assumed that methoxyphenyl end-groups and dioxaborine rings in 4 are simultaneously oxidized and reduced, respectively, at the same potential.

### 2.2. Comparison of Photophysical Properties

The electronic absorption spectra of compounds X, Y, Z and 4 were recorded in DCM solutions (FIG. 2) and the spectroscopic data are reported in Table 2. The spectra consist mainly of one intense transition band at low energy (450-550 nm) attributed to a strongly allowed π-π* transition. A second electronic transition band of much lower intensity appears as a shoulder at higher energy (<400 nm, vide infra). The spectra of compounds X, Y and Z display identical shape of the absorption profiles, they only differ by the position of the bands. In contrast, compound 4 exhibits a low-energy absorption band with very different shape, full width at half maximum, and intensity. The molar absorption coefficient determined for 4 is twice as high as that of compounds X, Y and Z. The more complex shape of the absorption band is likely to stem from intramolecular exciton coupling between the two curcuminoid chromophores.

Compounds X, Y, Z and 4 are fluorescent in the visible region (540-575 nm) upon excitation into the low-energy transition band and exhibit fluorescence quantum yields ranging from 44 to 61% in DCM. In agreement with electronic absorption data, an increase of the donor strength causes a red-shift of the fluorescence emission from 538 nm (X) to 574 nm (4). It is worth noting that the highest value of 0c (61%) is obtained for complex 4, giving a high brightness value of ca. 87000 M⁻¹ cm⁻¹.

TABLE 2
Spectroscopic data and photophysical properties of all borondifluoride compounds
solvated in DCM and as particles in water at room temperature^{a}

| | UV-vis | | Fluorescence | | | | | | | TPEF | | |
|---|---|---|---|---|---|---|---|---|---|---|---|---|
| compound | λ^{abs} | εₘₐₓ | λ^{em} | Δ ν_{ST} | Φ_{f} | B¹ | τ_{f} | k_{f} | kₙᵣ | λ²ₘₐₓ | σ^{TPA} | B² |
| X (DCM) | 488 | 75480 | 538 | 1904 | 0.44 | 33211 | 1.30 | 3.4 | 4.3 | 770 | 155 | 68 |
| Y (DCM) | 524 | 84860 | 566 | 1416 | 0.52 | 44127 | 1.72 | 3.0 | 2.8 | 810 | 248 | 129 |
| Z (DCM) | 511 | 74510 | 561 | 1957 | 0.46 | 34275 | 1.74 | 2.6 | 3.1 | 790 | 208 | 96 |
| 4 (DCM) | 529 | 143140 | 574 | 1421 | 0.61 | 87315 | 1.69 | 3.6 | 2.3 | 800 | 513 | 313 |
| X (water) | 451 | 34170 | 710 | 8088^{b} | 0.06 | 2050 | 6.44 | 0.09 | 1.5 | 780 | 210 | 13 |
| Y (water) | 489 | 27380 | 717 | 6503^{b} | 0.035 | 783 | 1.32 | — | — | —^{d} | —^{d} | —^{d} |
| | | | | | | | 2.91^{c} | | | | | |
| Z (water) | 407 | 29500 | 711 | 10505^{b} | 0.015 | 443 | 2.13 | — | — | —^{d} | —^{d} | —^{d} |
| | | | | | | | 6.01^{c} | | | | | |
| 4 (water) | 468 | 55620 | 692 | 6917^{b} | 0.125 | 6952 | 1.89 | — | — | 850 | 560 | 70 |
| | | | | | | | 6.64^{c} | | | | | |

^{a}Absorption maximum wavelengths λ^{abs} (nm); Molar absorption coefficients at maximum εₘₐₓ (M⁻¹ cm⁻¹); Fluorescence maximum wavelengths λ^{em} (nm); Stokes shifts Δ ν_{ST} (cm⁻¹); Fluorescence quantum yields Φ_{f}; Brightness B = Φ_{f} × ε (M⁻¹ cm⁻¹); Fluorescence lifetimes τ_{f} (ns); Radiative k_{f} (10⁸ s⁻¹) and nonradiative kₙᵣ = (1 − Φ_{f})/τ_{f} (10⁸ s⁻¹) rate constants; Two-photon absorption maximum λ²ₘₐₓ (nm); Two-photon cross section σ^{TPA} (GM); Two-photon brightness B² = Φ_{f} × σ^{TPA} (GM).
^{b}Pseudo-Stokes shift determined using the maximum absorption.
^{c}A biexponential decay was found
^{d}Not determined due to high scattering of light with those particles.

Two-photon excited fluorescence emission and excitation spectra of X, Y, Z and 4 were recorded in the 700-1000 nm wavelength range using a femtosecond Ti-Sapphire pulsed laser source, according to the experimental protocol described by Webb et al. (C. Xu and W. W. Webb, J. Opt. Soc. Am. B, 1996, 13, 481-491) using coumarin-307 and rhodamine B as references (C. Xu and W. W. Webb, J. Opt. Soc. Am. B, 1996, 13, 481-491). The observation of a quadratic dependence of the fluorescence intensity versus incident laser power at several wavelengths unambiguously confirmed that the origin of the fluorescence emission can be assigned to a TPA process in DCM solution. In the experimental laser power range used for these measurements, it was checked that no saturation or photodegradation occurred. The two-photon excitation spectra of X, Y, Z and 4 in DCM are shown in FIG. 3 and the corresponding data are reported in Table 2. X has a TPA cross section (σ^{TPA}) of 155 GM at 770 nm while compound Y, containing stronger donating groups, has its maximum red-shifted to 810 nm with a larger σ^{TPA} value of ca. 248 GM. It is interesting to note that Z presents an intermediate σ^{TPA} value of 208 GM at 790 nm. For compound 4, as already observed for the molar absorption coefficient, the two-photon cross section is slightly more than twice (i.e. 513 GM) the value determined for Z. This gives a two-photon brightness of 313 GM, that is much higher than those obtained with the model borondifluoride complexes X, Y, Z.

### 2.3. Comparison of Solid-State Optical Properties

Solid-state particles were prepared by quickly adding a concentrated THF solution of the compound Y, Z and 4 into water according to the classical fast precipitation method (H. Kawai, H. S. Nalwa, H. Oikawa, S. Okada, H. Matsuda, N. Minami, A. Kakuda, K. Ono, A. Mukoh and H. Nakanishi, Jpn. J. Appl. Rhys., 1992, 31, L1132-L1134). The so-obtained suspensions enabled the measurement of the UV/visible absorption and fluorescence spectra of the aggregated molecules. The preparation of X has been reported by Felouat et al. (A. Felouat, A. D'Aléo and F. Fages, J. Org. Chem. 2013, 78, 4446-4455) and the resulting spectroscopic data are recalled here for the sake of comparison. The shape of the electronic absorption spectra of compounds X, Y Z, and 4 is strongly affected relative to the solution spectra. The absorption profiles of X, Y and 4 (FIGS. 4a, 4b, and 4d, respectively) are complex, broad, and their maximum presents a hypsochromic shift (ca. 30-60 nm). These observations show that excitonic interactions prevail in the condensed phase, which could be correlated with single-crystal X-ray diffraction data in the case of X. The absorption spectrum of Z displays a very different effect (FIGS. 4c). The low-energy transition is considerably blue-shifted (of ca. 96 nm) and has a narrower shape as compared to that of the previous curcuminoid particles. These spectroscopic features are the signature of H-aggregation in the solid state for compound Z. Noticeably, 4, also containing unsymmetrical curcuminoid chromophores, does not adopt such arrangement.

Particles of compounds Y, Z and 4 fluoresce in the NIR, from 692 to 717 nm (FIG. 4, Table 2). The spectra are considerably red-shifted (superior to 95 nm) as compared to those obtained in solution in the highly polar acetonitrile solvent. Such long emission wavelengths can be attributed to chromophore packing in the solid state, as shown by X-ray diffraction for X and by electronic absorption spectroscopy for the others (FIG. 4). The fluorescence quantum yields of X, Y and Z are found in the range of 1.5-6.0% which are significant values for aggregated organic dyes emitting in the NIR. Not surprisingly, the H-aggregated compound Z affords the lowest value of 1.5%. Remarkably, the Φ_{f} of dye 4 reaches a substantial value of 12.5% in the solid state, which makes compound 4 a very bright solid-state NIR fluorophore (brightness at 700 nm of 6952 M⁻¹ cm⁻¹). Actually, a rather large part of emitted photons by the four compounds have energies below 700 nm because the particle emission spectra are broad. However, it can be highlighted that, when only photons at longer wavelength than 700 nm are integrated, dye 4 remains the most luminescent borondifluoride complex of the series with a NIR luminescence quantum yield of ca. 6.5% while the other three dyes have quantum yields of 4.0, 1.5 and 1.0%, respectively.

In addition, two-photon properties of 4 could be measured and compared to the previously reported X particles. Noticeably, the Y and Z particles could not be measured due to the much higher light scattering in those samples which precluded obtaining reliable data. As observed in DCM solution and for X in water, the two-photon maximum of 4 does not overlap the maximum of the one photon absorption S₀-S₁ transition (FIG. 5) but it better matches the S₀-S₂ one (vide supra). This maximum is located at 850 nm with a two-photon cross section of ca. 560 GM. Such two-photon cross section value is 2.5 times higher than that of X which, associated to the higher fluorescence quantum yield of 4, results in a much higher two-photon brightness for 4 (more than 5 times greater than the one of X).

### 2.4. Conclusion

In compound 4, the absorption spectrum reveals that an intramolecular excitonic interaction exists in solution, showing that the two chromophoric units are not optically independent. Compound 4 displays a high fluorescence quantum yield, and a good value of the two-photon absorption cross section, which makes it an attractive fluorophore. Like compounds X, Y and Z, compound 4 experiences intermolecular interactions in the condensed phase. However, it exhibits the higher value of fluorescence quantum yield within the series investigated.

1. A compound of general formula (I):
Q²-Q¹-Q³ (I)
wherein
Q¹ represented by formula (II) 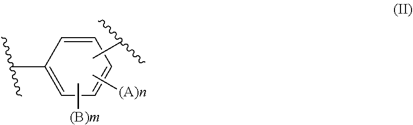
wherein
Q¹ is substituted with Q² and Q³,
A is a C₁-C₁₂ alkoxy group,
B is a difluoroboron beta-diketone of formula (III) 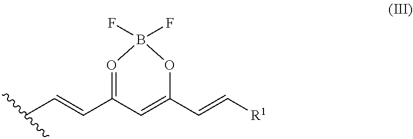
n is 0, 1, 2, 3 or 4,
m is 0 or 1; or
Q¹ is represented by formula (IV) 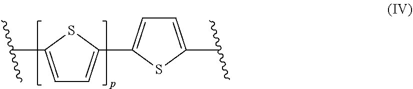
wherein
Q¹ is substituted with Q² and Q³,
p is 0, 1 or 2;
Q² is a difluoroboron beta-diketone of formula (V) 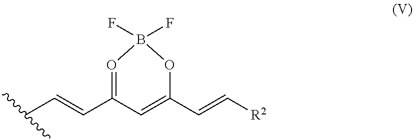
Q³ is a difluoroboron beta-diketone of formula (VI) 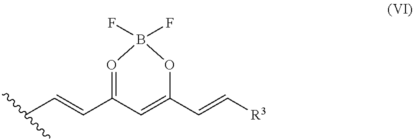
R¹, R² and R³, are each independently chosen among: 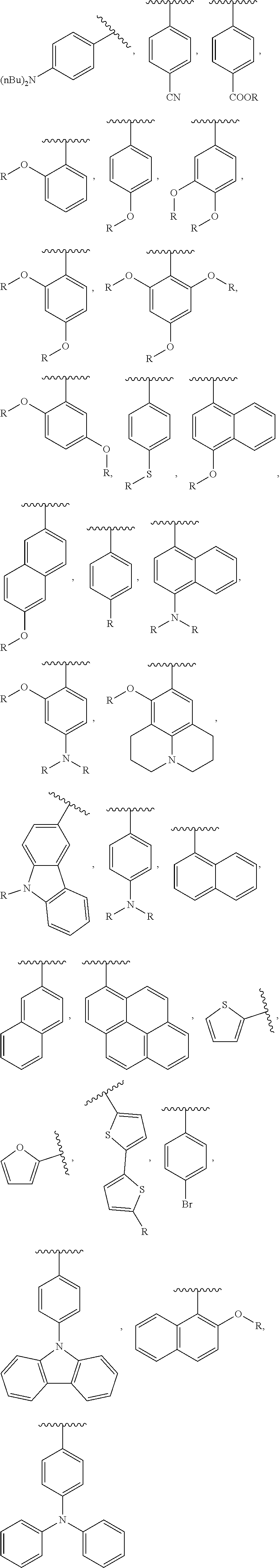
with R being each independently a hydrogen atom, a C₁-C₁₂ alkyl group, an C₆-C₁₀ aryl group, or a heteroaryl group, said alkyl, aryl and heteroaryl groups being optionally substituted.

2. The compound of formula (I) according to claim 1, wherein
Q¹ is represented by formula (IIa) 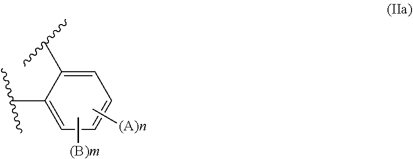
wherein
Q¹ is substituted with Q² and Q³ in ortho position,
A is a C₁-C₁₂ alkoxy group,
B is a difluoroboron beta-diketone of formula (III), 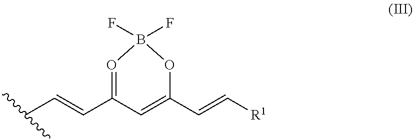
n is 0, 1, 2, 3 or 4,
m is 0 or 1; and
Q², Q³, R, R¹, R² and R³, are as defined in claim 1.

3. The compound of formula (I) according to claim 1, wherein
Q¹ is represented by formula (IIa) 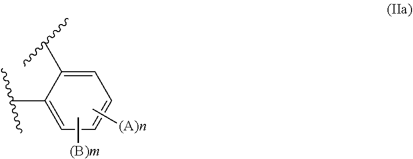
wherein
Q¹ is substituted with Q² and Q³ in ortho position,
n=m=0; and
Q² and Q³ are as defined in claim 1, with R² and R³ being each independently chosen among:

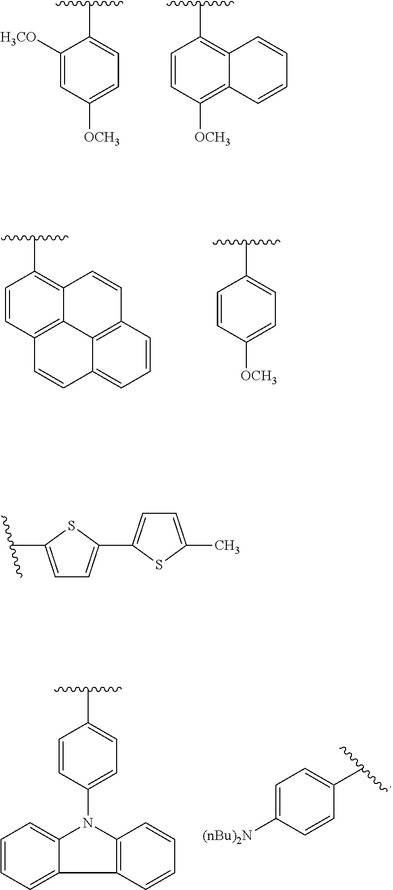

4. The compound of formula (I) according to claim 1, wherein
Q¹ is represented by formula (IIb) 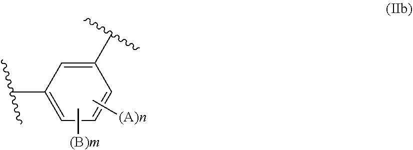
wherein
Q¹ is substituted with Q² and Q³ in meta position,
A is a C₁-C₁₂ alkoxy group,
B is a difluoroboron beta-diketone of formula (III) 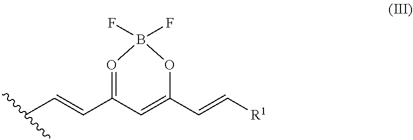
n is 0, 1, 2, 3 or 4,
m is 0 or 1; and
Q², Q³, R, R¹, R² and R³ are as defined in claim 1.

5. The compound of formula (I) according to claim 1, wherein
Q¹ is represented by formula (IIb) 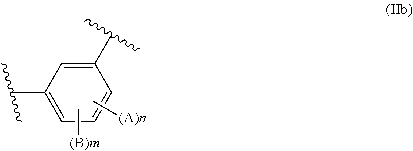
wherein
Q¹ is substituted with Q² and Q³ in meta position,
A is a C₁-C₁₂ alkoxy group,
B is a difluoroboron beta-diketone of formula (III) 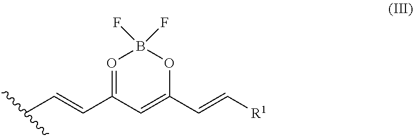
n is 0, 1, 2, 3 or 4,
m is 0 or 1; and
Q² and Q³ are as defined in claim 1, with R¹, R² and R³ being each independently chosen among:

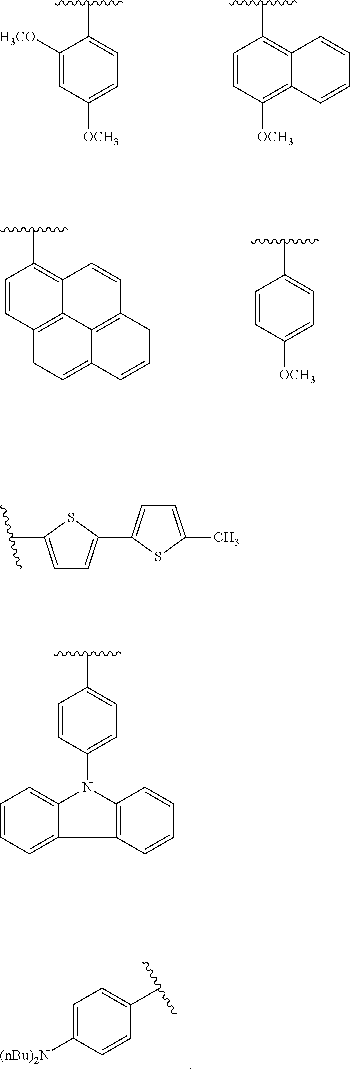

6. The compound of formula (I) according to claim 1, wherein
Q¹ is represented by formula (IIb) 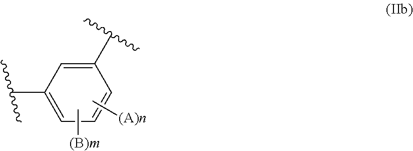
wherein
Q¹ is substituted with Q² and Q³ in meta position,
n=1, 2, 3 or 4,
m=0,
A is a C₁-C₁₂ alkoxy group; and
Q² and Q³ are as defined in claim 1, with R² and R³ being each independently chosen among:

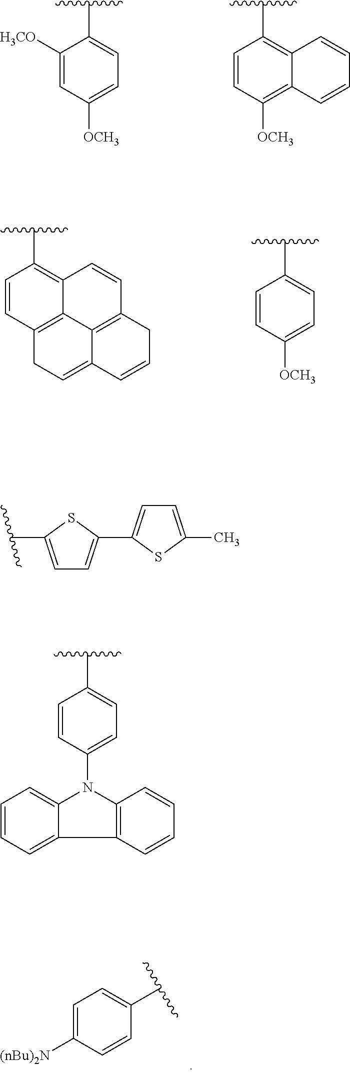

7. The compound of formula (I) according to claim 1, wherein
Q¹ is represented by formula (IIc) 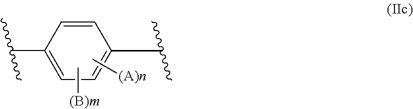
wherein
Q¹ is substituted with Q² and Q³ in para position,
A is a C₁-C₁₂ alkoxy group,
B is a difluoroboron beta-diketone of formula (III) 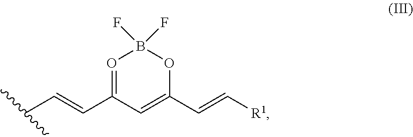
n is 0, 1, 2, 3 or 4,
m is 0 or 1; and
Q², Q³, R, R¹, R² and R³ are as defined in claim 1.

8. The compound of formula (I) according to claim 1, wherein
Q¹ is represented by formula (IIc) 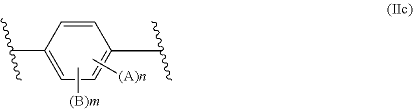
wherein
Q¹ is substituted with Q² and Q³ in para position,
A is a C₁-C₁₂ alkoxy group,
B is a difluoroboron beta-diketone of formula (III), 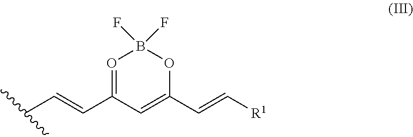
n is 0, 1, 2, 3 or 4,
m is 0 or 1;
Q² and Q³ are as defined in claim 1, with R¹, R² and R³ being each independently

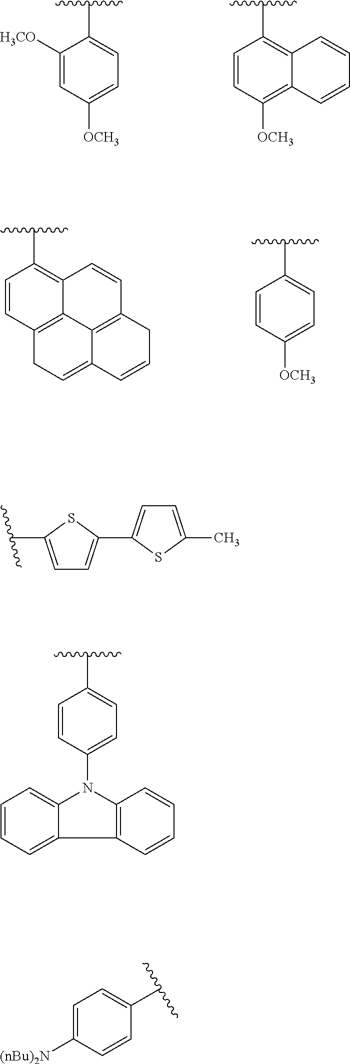

9. The compound of formula (I) according to claim 1, wherein
Q¹ is represented by formula (IIc) 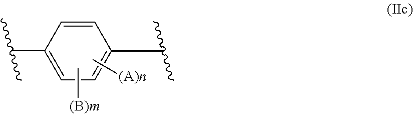
wherein
Q¹ is substituted with Q² and Q³ in para position,
A is a C₁-C₁₂ alkoxy group,
n is 0, 1, 2, 3 or 4,
m=0; and
Q² and Q³ are as defined in claim 1, with R² and R³ being each independently chosen among:

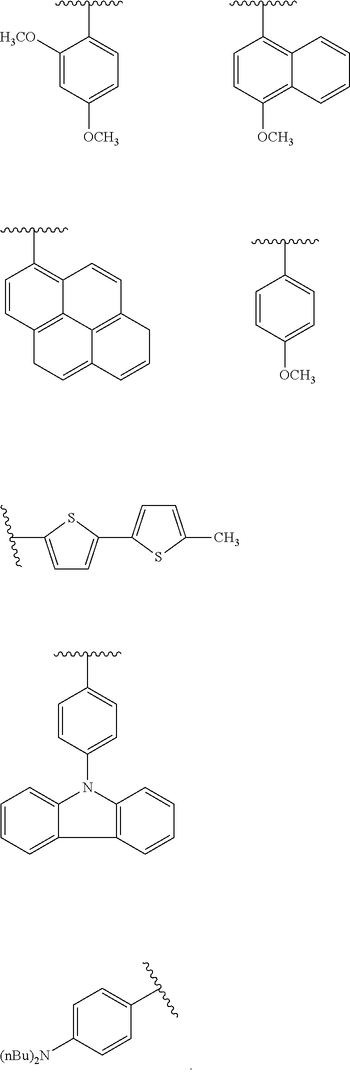

10. The compound of formula (I) according to claim 1, wherein
Q¹ is represented by formula (IV) 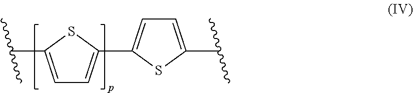
wherein
Q¹ is substituted with Q² and Q³,
p is 0, 1 or 2; and
Q², Q³, R² and R³ are as defined in claim 1.

11. The compound of formula (I) according to claim 1, wherein
Q¹ is represented by formula (IV) 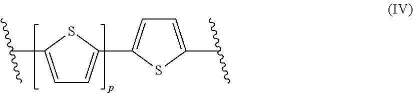
wherein
Q¹ is substituted with Q² and Q³,
p is 0, 1 or 2; and
Q² and Q³ are as defined in claim 1, with R² and R³ being each independently chosen among:

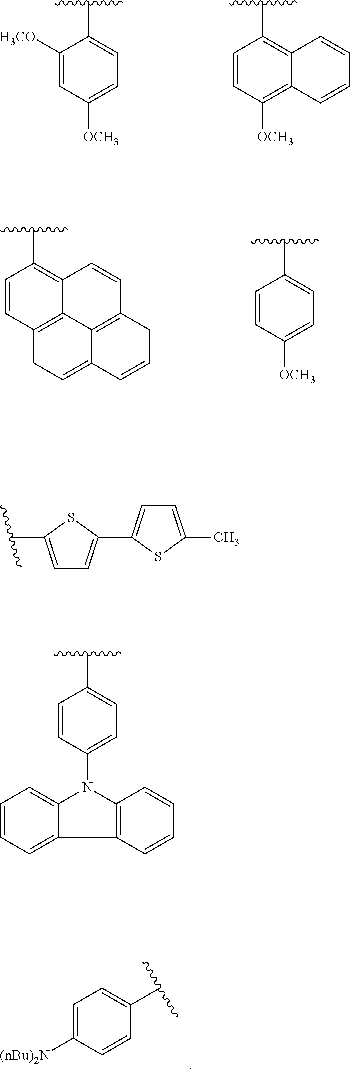

12. Use of a compound of formula (I) according to claim 1, as a fluorophore.

13. Use of a compound of formula (I) according to claim 1, in bioimaging, as sensors of volatile acid/base, in photodynamic therapy, in diagnosis of Alzheimer's disease, in theranostics, as optical sensors for anaerobic environment, in display and telecommunication technologies, in photovoltaics.
